(19) United States
(12) Patent Application Publication
(10) Pub. No.: US 2005/0159404 A1
(43) Pub. Date: Jul. 21, 2005

(54) Organic compound

(76) Inventors: Ming-Sing Si, San Franciso, CA (US); Dominic C. Borie, Palo Alto, CA (US); Bruce A. Reitz, Stanford, CA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/936,195

(22) Filed: Sep. 8, 2004

Related U.S. Application Data
(60) Provisional application No. 60/502,137, filed on Sep. 11, 2003.

(51) Int. Cl. A61K 31/553; A61K 38/13; A61K 31/407; A61K 31/405

(52) U.S. Cl. 514/211.08; 514/414; 514/11; 514/411

(57) ABSTRACT

A combination which comprises (a) a staurosporine derivative and (b) an agent effective in preventing, delaying or treating transplant rejection in which the active ingredients (a) and (b) are present in each case in free form or in the form of a pharmaceutically acceptable salt and optionally at least one pharmaceutically acceptable carrier; for simultaneous, separate or sequential use.

The invention relates to the use of a staurosporine derivative for the preparation of a medicament for delaying, preventing, or treating acute or chronic transplant rejection, to a combination which comprises (a) a staurosporine derivative and (b) an agent effective in preventing, delaying or treating transplant rejection and optionally at least one pharmaceutically acceptable carrier for simultaneous, separate or sequential use, in delaying, preventing, or treating acute or chronic transplant rejection; a pharmaceutical composition comprising such a combination; the use, of such a pharmaceutical composition or of such a combination for the preparation of a medicament, for delaying, preventing, or treating acute or chronic transplant rejection; a commercial package or product comprising such a combination as a combined preparation for simultaneous, separate or sequential use; and to a method of treatment of a patient, especially a recipient patient of organ, tissue or cell transplant.

Clinically used small molecule anti-rejection medications include cyclosporins, tacrolimus, sirolimus, mycophenalate mofetil and corticosteroids. Although these agents have improved short-term allograft survival by inhibiting acute rejection, they are also associated with toxicities that lead to significant morbidity and even mortality in allograft recipients. These shortcomings provide the impetus to discover and develop novel anti-rejection treatments that may improve or at least not increase the dosage of current anti-rejection regimens.

Unexpectedly, it has been found that a staurosporine derivative can be used as anti-transplant rejection drug.

Surprisingly also, it has been found that the effect, in treating or preventing acute or chronic transplant rejection, of a combination as defined herein is greater than the effects that can be achieved with either type of combination partner alone, i.e. greater than the effects of a monotherapy using only one of the combination partners (a) a staurosporine derivative or (b) an agent effective in preventing, delaying or treating transplant rejection as defined herein.

Hence, the present invention relates to a method of delaying, preventing or treating acute or chronic transplant rejection in a recipient patient comprising administering to said patient a combination which comprises (a) a staurosporine derivative and (b) an agent effective in preventing, delaying or treating transplant rejection, in a quantity which is jointly therapeutically effective against acute or chronic transplant rejection and in which the compounds can also be present in the form of their pharmaceutically acceptable salts.

Furthermore, the present invention pertains to a combination, such as a combined preparation or a pharmaceutical composition, which comprises (a) a staurosporine derivative and (b) a chemotherapeutic agent selected from the agents effective in preventing, delaying or treating transplant rejection, wherein the active ingredients are present in each case in free form or in the form of a pharmaceutically acceptable salt, and optionally at least one pharmaceutically acceptable carrier; for simultaneous, separate or sequential use.

The term “a combined preparation”, as used herein defines especially a “kit of parts” in the sense that the combination partners (a) and (b) as defined above can be dosed, independently of each other or by use of different fixed combinations with distinguished amounts of the combination partners (a) and (b), i.e., simultaneously or at different time points. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the combination partners (a) and (b). The ratio of the total amounts of the combination partner (a) to the combination partner (b) to be administered in the combined preparation can be varied, e.g. in order to cope with the needs of a patient sub-population to be treated or the needs of the single patient which different needs can be due to age, sex, body weight, etc. of the patients. Preferably, there is at least one beneficial effect, e.g., additive effect or a mutual enhancing of the effect of the combination partners (a) and (b), in particular a synergism, e.g. a more than additive effect, additional advantageous effects, less side effects, a combined therapeutic effect in a non-effective dosage of one or both of the combination partners (a) and (b), and very preferably a strong synergism of the combination partners (a) and (b).

The term “treatment” comprises the administration of the combination partners to a recipient patient of organ, tissue or cell transplant in need of such treatment with the aim to have the effect of preventing, delaying, hampering transplant rejection.

The term “treating” means that any transplant rejection mechanism undergoing will be stopped or slowed down.

The term “preventing” means that the administration of a staurosporine derivative or of the combination partners to a recipient patient of organ, tissue or cell transplant will hamper any transplant rejection processes before it starts.

The term “delaying” as used herein means that transplant rejection would at least be slowed down by the treatment and that patients exhibit higher transplant survival rates or for longer period of time than patients not being treated or being treated with the monotherapy.

The term “a staurosporine derivative” according to the present invention relates to staurosporine derivatives of formula A, B, C, D, I, II, III, IV, V, VI.

The “staurosporine derivatives” of the present invention have the following formulas, 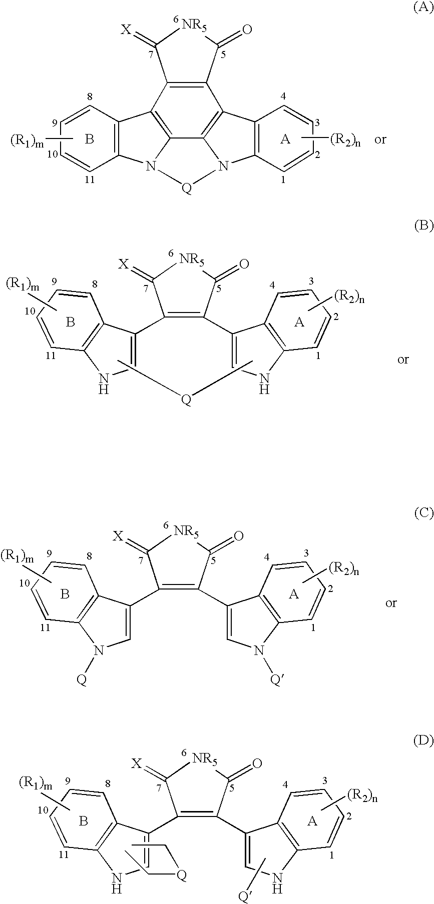
wherein R₁, and R₂ are, independently of one another, unsubstituted or substituted alkyl, hydrogen, halogen, hydroxy, etherified or esterified hydroxy, amino, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl or N-mono- or N,N-di-substituted aminosulfonyl;
n and m are, independently of one another, a number from and including 0 to and including 4;
R₅ is hydrogen, an aliphatic, carbocyclic, or carbocyclic-aliphatic radical with up to 29 carbon atoms in each case, or a heterocyclic or heterocyclic-aliphatic radical with up to 20 carbon atoms in each case, and in each case up to 9 heteroatoms, or acyl with up to 30 carbon atoms;
X stands for 2 hydrogen atoms; for 1 hydrogen atom and hydroxy; for O; or for hydrogen and lower alkoxy;
Q and Q′ are independently a pharmaceutically acceptable organic bone or hydrogen, halogen, hydroxy, etherified or esterified hydroxy, amino, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl or N-mono- or N,N-di-substituted aminosulfonyl;
or a salt thereof, if at least one salt-forming group is present, or hydrogenated derivative thereof, for the preparation of a pharmaceutical composition for the treatment of acute or chronic transplant rejection.

The term “organic bone” as used herein refers to a pharmacologically acceptable organic chemical structure, such as but not limited to hydrocarbyl radical or an acyl radical Ac, which radicals preferably have a maximum of 30 carbon atoms.

The hydrocarbyl radical (hydrocarbon radical) is an acyclic (aliphatic), carbocyclic or carbocyclic-acyclic hydrocarbon radical having a maximum total number of carbon atoms of preferably 30 and, especially, 18, which may be saturated or unsaturated, unsubstituted or substituted. It may also contain instead of one, two or more carbon atoms the same or different hetero atoms, such as, especially, oxygen, sulphur and nitrogen, in the acyclic and/or cyclic moiety; in the latter case it is referred to as a heterocyclic radical (heterocyclyl radical) or a heterocyclic-acyclic radical.

Unsaturated radicals are those that contain one or more, especially conjugated and/or isolated, multiple bonds (double and/or triple bonds). The term “cyclic radicals” also includes aromatic radicals, for example those in which at least one 6-membered carbocyclic ring or one 5- to 8-membered heterocyclic ring contains the maximum number of non-cumulated double bonds. Carbocyclic radicals in which at least one ring is in the form of a 6-membered aromatic ring (that is to say a benzene ring) are referred to as aryl radicals.

An acyclic unsubstituted hydrocarbon radical is especially a straight or branched lower alkyl, lower alkenyl, lower alkadienyl or lower alkynyl radical. In corresponding unsaturated radicals, the double bond is located especially in a position higher than the .alpha.-position to the free valency.

A carbocyclic hydrocarbon radical is especially a mono-, bi- or polycyclic cycloalkyl, cycloalkenyl or cycloalkadienyl radical, or a corresponding aryl radical. Preferred are radicals having a maximum of 14, especially 12, ring carbon atoms and having 3- to 8-membered, preferably 5- to 7-membered, especially 6-membered, rings; they may also carry one or more, for example two, acyclic radicals, for example those mentioned above, and especially lower alkyl radicals, or other carbocyclic radicals. Carbocyclic-acyclic radicals are those in which an acyclic radical, especially one having a maximum of 10, preferably a maximum of 6, carbon atoms, such as, especially, methyl, ethyl or vinyl, carries one or more of the carbocyclic, optionally aromatic radicals defined above. Mention is made especially of cycloalkyl-lower alkyl and aryl-lower alkyl radicals, and also analogues thereof unsaturated in the ring and/or chain, that carry the ring at the terminal carbon atom of the chain.

Linkers between the acyclic (aliphatic) or carbocyclic radicals may be selected from, but not limited to, straight or branched lower alkyl, lower alkenyl, lower alkadienyl or lower alkynyl radical, etherified or esterified hydroxy, amino, —O—, —S—, carbonyl, carbonyldioxy, —NO—, —SO—, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl or N-mono- or N,N-di-substituted aminosulfonyl.

What is important according to the present invention, is that the STAUROSPORINE DERIVATIVES have at least in common the staurosporine bone represented in the formula A, B, C, D.

The invention relates in particular to the use of staurosporines derivatives of formula, 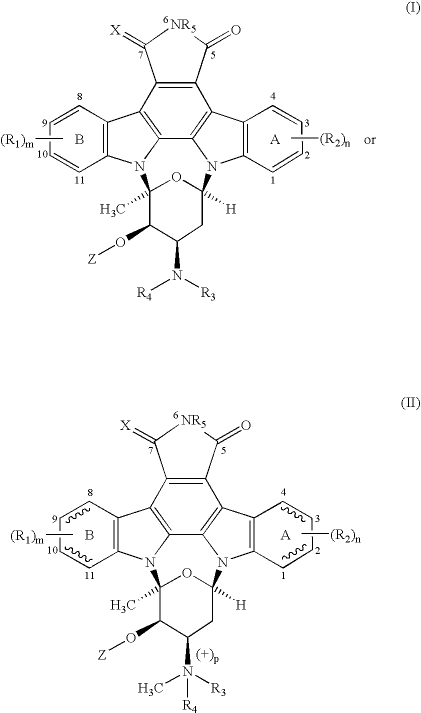
(II) which is the partially hydrogenated derivative of compound (I), 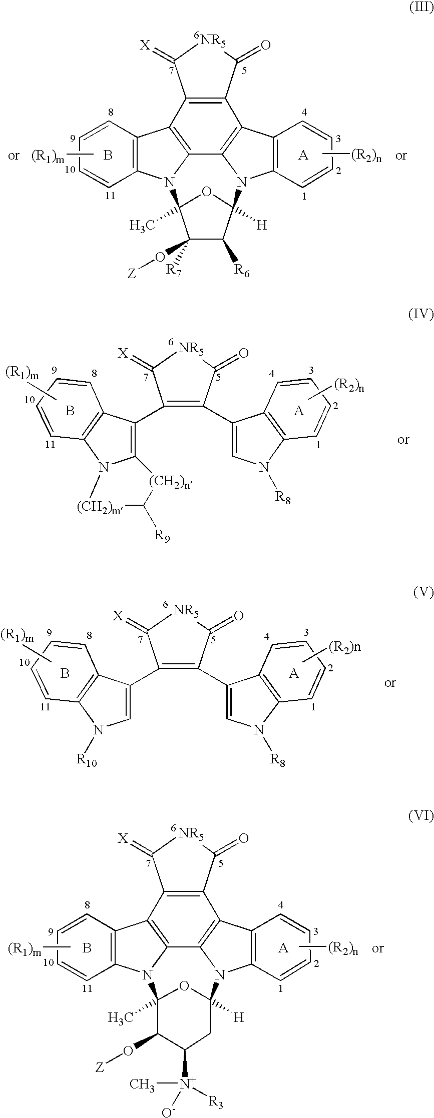
wherein R₁ and R₂, are, independently of one another, unsubstituted or substituted alkyl, hydrogen, halogen, hydroxy, etherified or esterified hydroxy, amino, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl or N-mono- or N,N-di-substituted aminosulfonyl;
n and m are, independently of one another, a number from and including 0 to and including 4;
n′ and m′ are, independently of one another, a number from and including 0 to and including 4;
R₃, R₄, R₈ and R₁₀ are, independently of one another, hydrogen, —O—, acyl with up to 30 carbon atoms, an aliphatic, carbocyclic, or carbocyclic-aliphatic radical with up to 29 carbon atoms in each case, a heterocyclic or heterocyclic-aliphatic radical with up to 20 carbon atoms in each case, and in each case up to 9 heteroatoms, wherein R₄ may also be absent;
if R₃ is acyl with up to 30 carbon atoms and R₄ is not an acyl;
p is 0 if R₄ is absent, or is 1 if R₃ and R₄ are both present and in each case are one of the aforementioned radicals;
R₅ is hydrogen, an aliphatic, carbocyclic, or carbocyclic-aliphatic radical with up to 29 carbon atoms in each case, or a heterocyclic or heterocyclic-aliphatic radical with up to 20 carbon atoms in each case, and in each case up to 9 heteroatoms, or acyl with up to 30 carbon atoms;
R₇, R₆ and R₉ are acyl or -(lower alkyl)-acyl, unsubstituted or substituted alkyl, hydrogen, halogen, hydroxy, etherified or esterified hydroxy, amino, mono- or disubstituted amino, cyano, nitro, mercapto, substituted mercapto, carboxy,carbonyl, carbonyldioxy, esterified carboxy, carbamoyl, N-mono- or N,N-di-substituted carbamoyl, sulfo, substituted sulfonyl, aminosulfonyl or N-mono- or N,N-di-substituted aminosulfonyl;
X stands for 2 hydrogen atoms; for 1 hydrogen atom and hydroxy; for O; or for hydrogen and lower alkoxy;
Z stands for hydrogen or lower alkyl;
and either the two bonds characterised by wavy lines are absent in ring A and replaced by 4 hydrogen atoms, and the two wavy lines in ring B each, together with the respective parallel bond, signify a double bond;
or the two bonds characterised by wavy lines are absent in ring B and replaced by a total of 4 hydrogen atoms, and the two wavy lines in ring A each, together with the respective parallel bond, signify a double bond;
or both in ring A and in ring B all of the 4 wavy bonds are absent and are replaced by a total of 8 hydrogen atoms;
or a salt thereof, if at least one salt-forming group is present for the preparation of a pharmaceutical composition for the treatment of acute or chronic transplant rejection.

The general terms and definitions used preferably have hereinbefore and hereinafter the following meanings:

The prefix “lower” indicates that the associated radical preferably has up to and including a maximum of 7 carbon atoms, especially up to and including a maximum of 4 carbon atoms.

Lower alkyl is especially methyl, ethyl, n-propyl, isopropyl, n-butyl, isobutyl, sec-butyl, or tert-butyl, and also pentyl, hexyl, or heptyl.

Unsubstituted or substituted alkyl is preferably C₁-C₂₀alkyl, especially lower alkyl, typically methyl, ethyl, n-propyl, isopropyl, n-butyl, isobutyl, sec-butyl, or tert-butyl, which is unsubstituted or substituted especially by halogen, such as fluorine, chlorine, bromine, or iodine, C₆-C₁₄aryl, such as phenyl or naphthyl, hydroxy, etherified hydroxy, such as lower alkoxy, phenyl-lower alkoxy or phenyloxy, esterified hydroxy, such as lower alkanoyloxy or benzoyloxy, amino, mono- or disubstituted amino, such as lower alkylamino, lower alkanoylamino, phenyl-lower alkylamino, N,N-di-lower alkylamino, N,N-di-(phenyl-lower alkyl)amino, cyano, mercapto, substituted mercapto, such as lower alkylthio, carboxy, esterified carboxy, such as lower alkoxycarbonyl, carbamoyl, N-mono- or N,N-disubstituted carbamoyl, such as N-lower alkylcarbamoyl or N,N-di-lower alkylcarbamoyl, sulfo, substituted sulfo, such as lower alkanesulfonyl or lower alkoxysulfonyl, aminosulfonyl or N-mono- or N,N-disubstituted aminosulfonyl, such as N-lower alkylaminosulfonyl or N,N-di-lower alkylaminosulfonyl.

Halogen is preferably fluorine, chlorine, bromine, or iodine, especially fluorine or chlorine.

Etherified hydroxy is especially lower alkoxy, C₆-C₁₄aryloxy, such as phenyloxy, or C₆-C₁₄aryl-lower alkoxy, such as benzyloxy.

Esterified hydroxy is preferably lower alkanoyloxy or C₆-C₁₄arylcarbonyloxy, such as benzoyloxy.

Mono- or disubstituted amino is especially amino monosubstituted or disubstituted by lower alkyl, C₆-C₁₄aryl, C₆-C₁₄aryl-lower alkyl, lower alkanoyl, or C₆-C₁₂arylcarbonyl.

Substituted mercapto is especially lower alkylthio, C₆-C₁₄arylthio, C₆-C₁₄aryl-lower alkylthio, lower alkanoylthio, or C₆-C₁₄aryl-lower alkanoylthio.

Esterified carboxy is especially lower alkoxycarbonyl, C₆-C₁₄aryl-lower alkoxycarbonyl or C₆-C₁₄aryloxycarbonyl.

N-Mono- or N,N-disubstituted carbamoyl is especially carbamoyl N-monosubstituted or N,N-disubstituted by lower alkyl, C₆-C₁₄aryl or C₆-C₁₄aryl-lower alkyl.

Substituted sulfonyl is especially C₆-C₁₄arylsulfonyl, such as toluenesulfonyl, C₆-C₁₄aryl-lower alkanesulfonyl or lower alkanesulfonyl.

N-Mono- or N,N-disubstituted aminosulfonyl is especially aminosulfonyl N-monosubstituted or N,N-disubstituted by lower alkyl, C₆-C₁₄aryl or C₆-C₁₄aryl-lower alkyl.

C₆-C₁₄Aryl is an aryl radical with 6 to 14 carbon atoms in the ring system, such as phenyl, naphthyl, fluorenyl, or indenyl, which is unsubstituted or is substituted especially by halogen, such as fluorine, chlorine, bromine, or iodine, phenyl or naphthyl, hydroxy, lower alkoxy, phenyl-lower alkoxy, phenyloxy, lower alkanoyloxy, benzoyloxy, amino, lower alkylamino, lower alkanoylamino, phenyl-lower alkylamino, N,N-di-lower alkylamino, N,N-di-(phenyl-lower alkyl)amino, cyano, mercapto, lower alkylthio, carboxy, lower alkoxycarbonyl, carbamoyl, N-lower alkylcarbamoyl, N,N-di-lower alkylcarbamoyl, sulfo, lower alkanesulfonyl, lower alkoxysulfonyl, aminosulfonyl, N-lower alkylaminosulfonyl, or N,N-di-lower alkylaminosulfonyl.

The indices n and m are in each case preferably 1, 2 or especially 0. In general, compounds of formula I in which n and m are in each case 0 (zero) are especially preferred.

An aliphatic carbohydrate radical with up to 29 carbon atoms R₃, R₄, R₈ or R₁₀, which is substituted by acyclic substituents and preferably has a maximum of 18, especially a maximum of 12, and as a rule not more than 7 carbon atoms, may be saturated or unsaturated and is especially an unsubstituted or a straight-chain or branched lower alkyl, lower alkenyl, lower alkadienyl, or lower alkinyl radical substituted by acyclic substituents. Lower alkyl is, for example, methyl, ethyl, n-propyl, isopropyl, n-butyl, isobutyl, sec-butyl or tert-butyl, and also n-pentyl, isopentyl, n-hexyl, isohexyl and n-heptyl; lower alkenyl is, for example, allyl, propenyl, isopropenyl, 2- or 3-methallyl and 2- or 3-butenyl; lower alkadienyl is, for example, 1-penta-2,4-dienyl; lower alkinyl is, for example, propargyl or 2-butinyl. In corresponding unsaturated radicals, the double bond is especially located in a position higher than the □-position in relation to the free valency. Substituents are especially the acyl radicals defined hereinbelow as substituents of R, preferably free or esterified carboxy, such as carboxy or lower alkoxycarbonyl, cyano or di-lower alkylamino.

A carbocyclic or carbocyclic-aliphatic radical R₃, R₄, R₈ or R₁₀ with up to 29 carbon atoms in each case is especially an aromatic, a cycloaliphatic, a cycloaliphatic-aliphatic, or an aromatic-aliphatic radical which is either present in unsubstituted form or substituted by radicals referred to hereinbelow as substituents of R°. An aromatic radical (aryl radical) R₃ or R₄ is most especially a phenyl, also a naphthyl, such as 1- or 2-naphthyl, a biphenylyl, such as especially 4-biphenylyl, and also an anthryl, fluorenyl and azulenyl, as well as their aromatic analogues with one or more saturated rings, which is either present in unsubstituted form or substituted by radicals referred to hereinbelow as substituents of R°. Preferred aromatic-aliphatic radicals are aryl-lower alkyl- and aryl-lower alkenyl radicals, e.g. phenyl-lower alkyl or phenyl-lower alkenyl with a terminal phenyl radical, such as for example benzyl, phenethyl, 1-, 2-, or 3-phenylpropyl, diphenylmethyl (benzhydryl), trityl, and cinnamyl, and also 1- or 2-naphthylmethyl. Of aryl radicals carrying acyclic radicals, such as lower alkyl, special mention is made of o-, m- and p-tolyl and xylyl radicals with variously situated methyl radicals.

A cycloaliphatic radical R₃, R₄, R₈ or R₁₀ with up to 29 carbon atoms is especially a substituted or preferably unsubstituted mono-, bi-, or polycyclic cycloalkyl-, cycloalkenyl-, or cycloalkadienyl radical. Preference is for radicals with a maximum of 14, especially 12, ring-carbon atoms and 3- to 8-, preferably 5- to 7-, and most especially 6-member rings which can also carry one or more, for example two, aliphatic hydrocarbon radicals, for example those named above, especially the lower alkyl radicals, or other cycloaliphatic radicals. Preferred substituents are the acyclic substituents named hereinbelow for R°.

A cycloaliphatic-aliphatic radical R₃, R₄, R₈ or R₁₀ with up to 29 carbon atoms is a radical in which an acyclic radical, especially one with a maximum of 7, preferably a maximum of 4 carbon atoms, such as especially methyl, ethyl, and vinyl, carries one or more cycloaliphatic radicals as defined hereinabove. Special mention is made of cycloalkyl-lower alkyl radicals, as well as their analogues which are unsaturated in the ring and/or in the chain, but are non-aromatic, and which carry the ring at the terminal carbon atom of the chain. Preferred substituents are the acyclic substituents named herein below for R°.

Heterocyclic radicals R₃, R₄, R₈ or R₁₀ with up to 20 carbon atoms each and up to 9 heteroatoms each are especially monocyclic, but also bi- or polycyclic, aza-, thia-, oxa-, thiaza-, oxaza-, diaza-, triaza-, or tetrazacyclic radicals of an aromatic character, as well as corresponding heterocyclic radicals of this type which are partly or most especially wholly saturated, these radicals—if need be—possibly carrying further acyclic, carbocyclic, or heterocyclic radicals and/or possibly mono-, di-, or polysubstituted by functional groups, preferably those named hereinabove as substituents of aliphatic hydrocarbon radicals. Most especially they are unsubstituted or substituted monocyclic radicals with a nitrogen, oxygen, or sulfur atom, such as 2-aziridinyl, and especially aromatic radicals of this type, such as pyrryl, for example 2-pyrryl or 3-pyrryl, pyridyl, for example 2-, 3-, or 4-pyridyl, and also thienyl, for example 2- or 3-thienyl, or furyl, for example 2-furyl; analogous bicyclic radicals with an oxygen, sulfur, or nitrogen atom are, for example, indolyl, typically 2- or 3-indolyl, quinolyl, typically 2- or 4-quinolyl, isoquinolyl, typically 3- or 5-isoquinolyl, benzofuranyl, typically 2-benzofuranyl, chromenyl, typically 3-chromenyl, or benzothienyl, typically 2- or 3-benzothienyl; preferred monocyclic and bicyclic radicals with several heteroatoms are, for example, imidazolyl, typically 2- or 4-imidazolyl, pyrimidinyl, typically 2- or 4-pyrimidinyl, oxazolyl, typically 2-oxazolyl, isoxazolyl, typically 3-isoxazolyl, or thiazolyl, typically 2-thiazolyl, and benzimidazolyl, typically 2-benzimidazolyl, benzoxazolyl, typically 2-benzoxazolyl, or quinazolyl, typically 2-quinazolinyl. Appropriate partially or, especially, completely saturated analogous radicals may also be considered, such as 2-tetrahydrofuryl, 2- or 3-pyrrolidinyl, 2-, 3-, or 4-piperidyl, and also 2- or 3-morpholinyl, 2- or 3-thiomorpholinyl 2-piperazinyl and N-mono- or N,N′-bis-lower alkyl-2-piperazinyl radicals. These radicals may also carry one or more acyclic, carbocyclic, or heterocyclic radicals, especially those mentioned hereinabove. The free valency of the heterocyclic radicals R₃ or R₄ must emanate from one of their carbon atoms. Heterocyclyl may be unsubstituted or substituted by one or more, preferably one or two, of the substituents named hereinbelow for R°.

Heterocyclic-aliphatic radicals R₃, R₄, R₈ or R₁o especially lower alkyl radicals, especially with a maximum of 7, preferably a maximum of 4 carbon atoms, for example those named hereinabove, which carry one, two, or more heterocyclic radicals, for example those named in the preceding paragraph, the heterocyclic ring possibly being linked to the aliphatic chain also by one of its nitrogen atoms. A preferred heterocyclic-aliphatic radical R₁ is, for example, imidazol-1-ylmethyl, 4-methylpiperazin-1-ylmethyl, piperazin-1-ylmethyl, 2-(morpholin-4-yl)ethyl and also pyrid-3-ylmethyl. Heterocyclyl may be unsubstituted or substituted by one or more, preferably one or two, of the substituents named herein below for R°.

A heteroaliphatic radical R₃, R₄, R₈ or R₁₀ with up to 20 carbon atoms each and up to 10 heteroatoms each is an aliphatic radical which, instead of one, two, or more carbon atoms, contains identical or different heteroatoms, such as especially oxygen, sulfur, and nitrogen. An especially preferred arrangement of a heteroaliphatic radical R₁ takes the form of oxa-alkyl radicals in which one or more carbon atoms are replaced in a preferably linear alkyl by oxygen atoms preferably separated from one another by several (especially 2) carbon atoms so that they form a repeating group, if need be multi-repeating group (O—CH₂—CH₂—)_{q}, wherein q=1 to 7.

Especially preferred as R₃, R₄, R₈ or R₁₀, apart from acyl, is lower alkyl, particlularly methyl or ethyl; lower alkoxycarbonyl-lower alkyl, especially methoxycarbonylmethyl or 2-(tert-butoxycarbonyl)ethyl; carboxy-lower alkyl, especially carboxymethyl or 2-carboxyethyl; or cyano-lower alkyl, especially 2-cyanoethyl.

An acyl radical R₃, R₄, R₆, R₇, R₈, R₉, or R₁₀ with up to 30 carbon atoms derives from a carboxylic acid, functionally modified if need be, an organic sulfonic acid, or a phosphoric acid, such as pyro- or orthophosphoric acid, esterified if need be.

An acyl designated Ac¹ and derived from a carboxylic acid, functionally modified if need be, is especially one of the subformula Y—C(═W)—, wherein W is oxygen, sulfur, or imino and Y is hydrogen, hydrocarbyl R° with up to 29 carbon atoms, hydrocarbyloxy R°—O—, an amino group or a substituted amino group, especially one of the formula R°HN— or R°R°N— (wherein the R° radicals may be identical or different from one another).

The hydrocarbyl (hydrocarbon radical) R° is an acyclic (aliphatic), carbocyclic, or carbocyclic-acyclic hydrocarbon radical, with up to 29 carbon atoms each, especially up to 18, and preferably up to 12 carbon atoms, and is saturated or unsaturated, unsubstituted or substituted. Instead of one, two, or more carbon atoms, it may contain identical or different heteroatoms, such as especially oxygen, sulfur, and nitrogen in the acyclic and/or cyclic part; in the latter case, it is described as a heterocyclic radical (heterocyclyl radical) or a hetero-cyclic-acyclic radical.

Unsaturated radicals are those, which contain one or more, especially conjugated and/or isolated, multiple bonds (double or triple bonds). The term cyclic radicals includes also aromatic and non-aromatic radicals with conjugated double bonds, for example those wherein at least one 6-member carbocyclic or a 5- to 8-member heterocyclic ring contains the maximum number of non-cumulative double bonds. Carbocyclic radicals, wherein at least one ring is present as a 6-member aromatic ring (i.e. a benzene ring), are defined as aryl radicals.

An acyclic unsubstituted hydrocarbon radical R° is especially a straight-chained or branched lower alkyl-, lower alkenyl-, lower alkadienyl-, or lower alkinyl radical. Lower alkyl R° is, for example, methyl, ethyl, n-propyl, isopropyl, n-butyl, isobutyl, sec-butyl or tert-butyl, and also n-pentyl, isopentyl, n-hexyl, isohexyl and n-heptyl; lower alkenyl is, for example, allyl, propenyl, isopropenyl, 2- or 3-methallyl and 2- or 3-butenyl; lower alkadienyl is, for example, 1-penta-2,4-dienyl; lower alkinyl is, for example, propargyl or 2-butinyl. In corresponding unsaturated radicals, the double bond is especially located in a position higher than the □-position in relation to the free valency.

A carbocyclic hydrocarbon radical R° is especially a mono-, bi-, or polycyclic cycloalkyl-, cycloalkenyl-, or cycloalkadienyl radical, or a corresponding aryl radical. Preference is for radicals with a maximum of 14, especially 12, ring-carbon atoms and 3- to 8-, preferably 5- to 7-, and most especially 6-member rings which can also carry one or more, for example two, acyclic radicals, for example those named above, especially the lower alkyl radicals, or other carbocyclic radicals. Carbocyclic-acyclic radicals are those in which an acyclic radical, especially one with a maximum of 7, preferably a maximum of 4 carbon atoms, such as especially methyl, ethyl and vinyl, carries one or more carbocyclic, if need be aromatic radicals of the above definition. Special mention is made of cycloalkyl-lower and aryl-lower alkyl radicals, as well as their analogues which are unsaturated in the ring and/or chain, and which carry the ring at the terminal carbon atom of the chain.

Cycloalkyl R° has most especially from 3 up to and including 10 carbon atoms and is, for example, cyclopropyl, cyclobutyl, cyclopentyl, cyclohexyl, cycloheptyl, and cyclooctyl, as well as bicyclo[2,2,2]octyl, 2-bicyclo[2,2,1]heptyl, and adamantyl, which may also be substituted by 1, 2, or more, for example lower, alkyl radicals, especially methyl radicals; cycloalkenyl is for example one of the monocyclic cycloalkyl radicals already named which carries a double bond in the 1-, 2-, or 3 position. Cycloalkyl-lower alkyl or -lower alkenyl is for example a -methyl, -1- or -2-ethyl, -1- or -2-vinyl, -1-, -2-, or -3-propyl or -allyl substituted by one of the above-named cycloalkyl radicals, those substituted at the end of the linear chain being preferred.

An aryl radical R° is most especially a phenyl, also a naphthyl, such as 1- or 2-naphthyl, a biphenylyl, such as especially 4-biphenylyl, and also an anthryl, fluorenyl and azulenyl, as well as their aromatic analogues with one or more saturated rings. Preferred aryl-lower alkyl and -lower alkenyl radicals are, for example, phenyl-lower alkyl or phenyl-lower alkenyl with a terminal phenyl radical, such as for example benzyl, phenethyl, 1-, 2-, or 3-phenylpropyl, diphenylmethyl (benzhydryl), trityl, and cinnamyl, and also 1- or 2-naphthylmethyl. Aryl may be unsubstituted or substituted.

Heterocyclic radicals, including heterocyclic-acyclic radicals, are especially monocyclic, but also bi- or polycyclic, aza-, thia-, oxa-, thiaza-, oxaza-, diaza-, triaza-, or tetrazacyclic radicals of an aromatic character, as well as corresponding heterocyclic radicals of this type which are partly or most especially wholly saturated; if need be, for example as in the case of the above-mentioned carbocyclic or aryl radicals, these radicals may carry further acyclic, carbocyclic, or heterocyclic radicals and/or may be mono-, di-, or polysubstituted by functional groups. The acyclic part in heterocyclic-acyclic radicals has for example the meaning indicated for the corresponding carbocyclic-acyclic radicals. Most especially they are unsubstituted or substituted monocyclic radicals with a nitrogen, oxygen, or sulfur atom, such as 2-aziridinyl, and especially aromatic radicals of this type, such as pyrrolyl, for example 2-pyrrolyl or 3-pyrrolyl, pyridyl, for example 2-, 3-, or 4-pyridyl, and also thienyl, for example 2- or 3-thienyl, or furyl, for example 2-furyl; analogous bicyclic radicals with an oxygen, sulfur, or nitrogen atom are, for example, indolyl, typically 2- or 3-indolyl, quinolyl, typically 2- or 4-quinolyl, isoquinolyl, typically 3- or 5-isoquinolyl, benzofuranyl, typically 2-benzofuranyl, chromenyl, typically 3-chromenyl, or benzothienyl, typically 2- or 3-benzothienyl; preferred monocyclic and bicyclic radicals with several heteroatoms are, for example, imidazolyl, typically 2-imidazolyl, pyrimidinyl, typically 2- or 4-pyrimidinyl, oxazolyl, typically 2-oxazolyl, isoxazolyl, typically 3-isoxazolyl, or thiazolyl, typically 2-thiazolyl, and benzimidazolyl, typically 2-benzimidazolyl, benzoxazolyl, typically 2-benzoxazolyl, or quinazolyl, typically 2-quinazolinyl. Appropriate partially or, especially, completely saturated analogous radicals may also be considered, such as 2-tetrahydrofuryl, 4-tetrahydrofuryl, 2- or 3-pyrrolidyl, 2-, 3-, or 4-piperidyl, and also 2- or 3-morpholinyl, 2- or 3-thiomorpholinyl, 2-piperazinyl, and N,N′-bis-lower alkyl-2-piperazinyl radicals. These radicals may also carry one or more acyclic, carbocyclic, or heterocyclic radicals, especially those mentioned hereinabove. Heterocyclic-acyclic radicals are especially derived from acyclic radicals with a maximum of 7, preferably a maximum of 4 carbon atoms, for example those named hereinabove, and may carry one, two, or more heterocyclic radicals, for example those named hereinabove, the ring possibly being linked to the aliphatic chain also by one of its nitrogen-atoms.

As already mentioned, a hydrocarbyl (including a heterocyclyl) may be substituted by one, two, or more identical or different substituents (functional groups); one or more of the following substituents may be considered: lower alkyl; free, etherified and esterified hydroxyl groups; carboxy groups and esterified carboxy groups; mercapto- and lower alkylthio- and, if need be, substituted phenylthio groups; halogen atoms, typically chlorine and fluorine, but also bromine and iodine; halogen-lower alkyl groups; oxo groups which are present in the form of formyl (i.e. aldehydo) and keto groups, also as corresponding acetals or ketals; azido groups; nitro groups; cyano groups; primary, secondary and preferably tertiary amino groups, amino-lower alkyl, mono- or disubstituted amino-lower alkyl, primary or secondary amino groups protected by conventional protecting groups (especially lower alkoxycarbonyl, typically tert-butoxycarbonyl) lower alkylenedioxy, and also free or functionally modified sulfo groups, typically sulfamoyl or sulfo groups present in free form or as salts. The hydrocarbyl radical may also carry carbamoyl, ureido, or guanidino groups, which are free or which carry one or two substituents, and cyano groups. The above use of the word “groups” is taken to imply also an individual group.

Halogen-lower alkyl contains preferably 1 to 3 halogen atoms; preferred is trifluoromethyl or chloromethyl.

An etherified hydroxyl group present in the hydrocarbyl as substituent is, for example, a lower alkoxy group, typically the methoxy-, ethoxy-, propoxy-, isopropoxy-, butoxy-, and tert-butoxy group, which may also be substituted, especially by (i) heterocyclyl, whereby heterocyclyl can have preferably 4 to 12 ring atoms, may be unsaturated, or partially or wholly saturated, is mono- or bicyclic, and may contain up to three heteroatoms selected from nitrogen, oxygen, and sulfur, and is most especially pyrrolyl, for example 2-pyrrolyl or 3-pyrrolyl, pyridyl, for example 2-, 3- or 4-pyridyl, and also thienyl, for example 2- or 3-thienyl, or furyl, for example 2-furyl, indolyl, typically 2- or 3-indolyl, quinolyl, typically 2- or 4-quinolyl, isoquinolyl, typically 3- or 5-isoquinolyl, benzofuranyl, typically 2-benzofuranyl, chromenyl, typically 3-chromenyl, benzothienyl, typically 2- or 3-benzothienyl; imidazolyl, typically 1- or 2-imidazolyl, pyrimidinyl, typically 2- or 4-pyrimidinyl, oxazolyl, typically 2-oxazolyl, isoxazolyl, typically 3-isoxazolyl, thiazolyl, typically 2-thiazolyl, benzimidazolyl, typically 2-benzimidazolyl, benzoxazolyl, typically 2-benzoxazolyl, quinazolyl, typically 2-quinazolinyl, 2-tetrahydrofuryl, 4-tetrahydrofuryl, 2- or 4-tetrahydropyranyl, 1-, 2- or 3-pyrrolidyl, 1-, 2-, 3-, or 4-piperidyl, 1-, 2- or 3-morpholinyl, 2- or 3-thiomorpholinyl, 2-piperazinyl or N,N′-bis-lower alkyl-2-piperazinyl; and also (ii) by halogen atoms, for example mono-, di-, or polysubstituted especially in the 2-position, as in the 2,2,2-trichloroethoxy, 2-chloroethoxy, or 2-iodoethoxy radical, or (iii) by hydroxy or (iv) lower alkoxy radicals, each preferably monosubstituted, especially in the 2-position, as in the 2-methoxyethoxy radical. Such etherified hydroxyl groups are also unsubstituted or substituted phenoxy radicals and phenyl-lower alkoxy radicals, such as especially benzyloxy, benzhydryloxy, and triphenylmethoxy (trityloxy), as well as heterocyclyloxy radicals, wherein heterocyclyl can have preferably 4 to 12 ring atoms, may be unsaturated, or partially or wholly saturated, is mono- or bicyclic, and may contain up to three heteroatoms selected from nitrogen, oxygen, and sulfur, and is most especially pyrrolyl, for example 2-pyrrolyl or 3-pyrrolyl, pyridyl, for example 2-, 3- or 4-pyridyl, and also thienyl, for example 2- or 3-thienyl, or furyl, for example 2-furyl, indolyl, typically 2- or 3-indolyl, quinolyl, typically 2- or 4-quinolyl, isoquinolyl, typically 3- or 5-isoquinolyl, benzofuranyl, typically 2-benzofuranyl, chromenyl, typically 3-chromenyl, benzothienyl, typically 2- or 3-benzothienyl; imidazolyl, typically 1- or 2-imidazolyl, pyrimidinyl, typically 2- or 4-pyrimidinyl, oxazolyl, typically 2-oxazolyl, isoxazolyl, typically 3-isoxazolyl, thiazolyl, typically 2-thiazolyl, benzimidazolyl, typically 2-benzimidazolyl, benzoxazolyl, typically 2-benzoxazolyl, quinazolyl, typically 2-quinazolinyl, 2-tetrahydrofuryl, 4-tetrahydrofuryl, 2- or 4-tetrahydropyranyl, 1-, 2- or 3-pyrrolidyl, 1-, 2-, 3-, or 4-piperidyl, 1-, 2- or 3-morpholinyl, 2- or 3-thiomorpholinyl, 2-piperazinyl or N,N′-bis-lower alkyl-2-piperazinyl; such as especially 2- or 4-tetrahydropyranyloxy.

Etherified hydroxyl groups in this context are taken to include silylated hydroxyl groups, typically for example tri-lower alkylsilyloxy, typically trimethylsilyloxy and dimethyl-tert-butylsilyloxy, or phenyldi-lower alkylsilyloxy and lower alkyl-diphenylsilyloxy.

An esterified hydroxyl group present in the hydrocarbyl as a substituent is, for example, lower alkanoyloxy.

A carboxyl group present in the hydrocarbyl as a substituent is one in which the hydrogen atom is replaced by one of the hydrogen radicals characterised hereinabove, preferably a lower alkyl- or phenyl-lower alkyl radical; an example of an esterified carboxyl group is lower alkoxycarbonyl or phenyl-lower alkoxycarbonyl substituted if need be in the phenyl part, especially the methoxy, ethoxy, tert-butoxy, and benzyloxycarbonyl group, as well as a lactonised carboxyl group.

A primary amino group —NH₂ as substituent of the hydrocarbyls may also be present in a form protected by a conventional protecting group. A secondary amino group carries, instead of one of the two hydrogen atoms, a hydrocarbyl radical, preferably an unsubstituted one, typically one of the above-named, especially lower alkyl, and may also be present in protected form.

A tertiary amino group present in the hydrocarbyl as substituent carries 2 different or, preferably, identical hydrocarbyl radicals (including the heterocyclic radicals), such as the unsubstituted hydrocarbyl radicals characterised hereinabove, especially lower alkyl.

A preferred amino group is one with the formula R₁₁(R₁₂)N—, wherein R₁₁ and R₁₂ are independently in each case hydrogen, unsubstituted acyclic C₁-C₇-hydrocarbyl (such as especially C₁-C₄alkyl or C₂-C₄alkenyl) or monocyclic aryl, aralkyl, or aralkenyl, substituted if necessary by C₁-C₄-alkyl, C₁-C₄-alkoxy, halogen, and/or nitro, and having a maximum of 10 carbon atoms, where the carbon-containing radicals may be interlinked through a carbon-carbon bond or an oxygen atom, a sulfur atom, or a nitrogen atom substituted if necessary by hydrocarbyl. In such a case, they form a nitrogen-containing heterocyclic ring with the nitrogen atom of the amino group. The following are examples of especially preferred disubstituted amino groups: di-lower alkylamino, typically dimethylamino or diethylamino, pyrrolidino, imidazol-1-yl, piperidino, piperazino, 4-lower alkylpiperazino, morpholino, thiomorpholino and piperazino or 4-methylpiperazino, as well as diphenylamino and dibenzylamino substituted if need be, especially in the phenyl part, for example by lower-alkyl, lower-alkoxy, halogen, and/or nitro; of the protected groups, especially lower alkoxy-carbonylamino, typically tert-butoxycarbonylamino, phenyl-lower alkoxycarbonylamino, typically 4-methoxybenzyloxycarbonylamino, and 9-fluorenylmethoxycarbonylamino.

Amino-lower alkyl is most especially substituted in the 1-position of the lower alkyl chain by amino and is especially aminomethyl.

Mono- or disubstituted amino-lower alkyl is amino-lower alkyl substituted by one or two radicals, wherein amino-lower alkyl is most especially substituted by amino in the 1-position of the lower alkyl chain and is especially aminomethyl; the amino substituents here are preferably (if 2 substituents are present in the respective amino group independently of one another) from the group comprising lower alkyl, such as especially methyl, ethyl or n-propyl, hydroxy-lower alkyl, typically 2-hydroxyethyl, C₃-C₈cycloalkyl, especially cyclohexyl, amino-lower alkyl, typically 3-aminopropyl or 4-aminobutyl, N-mono- or N,N-di(lower alkyl)-amino-lower alkyl, typically 3-(N,N-dimethylamino)propyl, amino, N-mono- or N,N-di-lower alkylamino and N-mono- or N,N-di-(hydroxy-lower alkyl)amino.

Disubstituted amino-lower alkyl is also a 5 or 6-membered, saturated or unsaturated heterocyclyl bonded to lower alkyl via a nitrogen atom (preferably in the 1-position) and having 0 to 2, especially 0 or 1, other heteroatoms selected from oxygen, nitrogen, and sulfur, which is unsubstituted or substituted, especially by one or two radicals from the group comprising lower alkyl, typically methyl, and also oxo. Preferred here is pyrrolidino (1-pyrrolidinyl), piperidino (1-piperidinyl), piperazino (1-piperazinyl), 4-lower alkylpiperazino, typically 4-methylpiperazino, imidazolino (1-imidazolyl), morpholino (4-morpholinyl), or also thiomorpholino, S-oxo-thiomorpholino, or S,S-dioxothiomorpholino.

Lower alkylenedioxy is especially methylenedioxy.

A carbamoyl group carrying one or two substituents is especially aminocarbonyl (carbamoyl) which is substitiuted by one or two radicals at the nitrogen; the amino substituents here are preferably (if 2 substituents are present in the respective amino group independently of one another) from the group comprising lower alkyl, such as especially methyl, ethyl or n-propyl, hydroxy-lower alkyl, typically 2-hydroxyethyl, C₃-C₈cycloalkyl, especially cyclohexyl, amino-lower alkyl, typically 3-aminopropyl or 4-aminobutyl, N-mono- or N,N-di(lower alkyl)-amino-lower alkyl, typically 3-(N,N-dimethylamino)propyl, amino, N-mono- or N,N-di-lower alkylamino and N-mono- or N,N-di-(hydroxy-lower alkyl)amino; disubstituted amino in aminocarbamoyl is also a 5 or 6-membered, saturated or unsaturated heterocyclyl with a bonding nitrogen atom and 0 to 2, especially 0 or 1, other heteroatoms selected from oxygen, nitrogen, and sulfur, which is unsubstituted or substituted, especially by one or two radicals from the group comprising lower alkyl, typically methyl, and also oxo. Preferred here is pyrrolidino (1-pyrrolidinyl), piperidino (1-piperidinyl), piperazino (1-piperazinyl), 4-lower al-kylpiperazino, typically 4-methylpiperazino, imidazolino (1-imidazolyl), morpholino (4-morpholinyl), or also thiomorpholino, S-oxo-thiomorpholino, or S,S-dioxothiomorpholino.

An acyl derived from an organic sulfonic acid, which is designated Ac², is especially one with the subformula R°—SO₂—, wherein R° is a hydrocarbyl as defined above in the general and specific meanings, the latter also being generally preferred here. Especially preferred is lower alkylphenylsulfonyl, especially 4-toluenesulfonyl.

An acyl derived from a phosphoric acid, esterified if necessary, which is designated Ac³, is especially one with the subformula R°O(R°O)P(═O)—, wherein the radicals R° are, independently of one another, as defined in the general and specific meanings indicated above.

Reduced data on substituents given hereinbefore and hereinafter are considered to be preferences.

Preferred compounds according to the invention are, for example, those wherein R⁰ has the following preferred meanings: lower alkyl, especially methyl or ethyl, amino-lower alkyl, wherein the amino group is unprotected or is protected by a conventional amino protecting group—especially by lower alkoxycarbonyl, typically tert-lower alkoxycarbonyl, for example tert-butoxycarbonyl—e.g. aminomethyl, R,S—,R— or preferably S-1-aminoethyl, tert-butoxycarbonylaminomethyl or R,S—,R—, or preferably S-1-(tert-butoxycarbonylamino)ethyl, carboxy-lower alkyl, typically 2-carboxyethyl, lower alkoxycarbonyl-lower alkyl, typically 2-(tert-butoxycarbonyl)ethyl, cyano-lower alkyl, typically 2-cyanoethyl, tetrahydropyranyloxy-lower alkyl, typically 4-(tetrahydropyranyl)-oxymethyl, morpholino-lower alkyl, typically 2-(morpholino)ethyl, phenyl, lower alkylphenyl, typically 4-methylphenyl, lower alkoxyphenyl, typically 4-methoxyphenyl, imidazolyl-lower alkoxyphenyl, typically 4-[2-(imidazol-1-yl)ethyl)oyxphenyl, carboxyphenyl, typically 4-carboxyphenyl, lower alkoxycarbonylphenyl, typically 4-ethoxycarbonylphenyl or 4-methoxyphenyl, halogen-lower alkylphenyl, typically 4-chloromethylphenyl, pyrrolidinophenyl, typically 4-pyrrolidinophenyl, imidazol-1-ylphenyl, typically 4-(imidazolyl-1-yl)phenyl, piperazinophenyl, typically 4-piperazinophenyl, (4-lower alkylpiperazino)phenyl, typically 4-(4-methylpiperazino)phenyl, morpholinophenyl, typically 4-morpholinophenyl, pyrrolidino-lower alkylphenyl, typically 4-pyrrolidinomethylphenyl, imidazol-1-yl-lower alkylphenyl, typically 4-(imidazolyl-1-ylmethyl)phenyl, piperazino-lower alkylphenyl, typically 4-piperazinomethylphenyl, (4-lower alkylpiperazinomethyl )-phenyl, typically 4-(4-methylpiperazinomethyl)phenyl, morpholino-lower alkylphenyl, typically 4-morpholinomethylphenyl, piperazinocarbonylphenyl, typically 4-piperazinocarbonylphenyl, or (4-lower alkyl-piperazino)phenyl, typically 4-(4-methylpiperazino)phenyl.

Preferred acyl radicals Ac¹ are acyl radicals of a carboxylic acid which are characterised by the subformula R°—CO—, wherein R° has one of the above general and preferred meanings of the hydrocarbyl radical R°. Especially preferred radicals R° here are lower alkyl, especially methyl or ethyl, amino-lower alkyl, wherein the amino group is unprotected or protected by a conventional amino protecting group, especially by lower alkoxycarbonyl, typically tert-lower alkoxycarbonyl, for example tert-butoxycarbonyl, e.g. aminomethyl, R,S—,R—, or preferably S- 1-aminoethyl, tert-butoxycarbonylaminomethyl or R,S—,R—, or preferably S-1-(tert-butoxycarbonylamino)ethyl, carboxy-lower alkyl, typically 2-carboxyethyl, lower alkoxycarbonyl-lower alkyl, typically 2-(tert-butoxycarbonyl)ethyl, tetrahydropyranyloxy-lower alkyl, typically 4-(tetrahydropyranyl)oxymethyl, phenyl, imidazolyl-lower alkoxyphenyl, typically 4-[2-(imidazol-1-yl)ethyl]oyxphenyl, carboxyphenyl, typically 4-carboxyphenyl, lower alkoxycarbonylphenyl, typically 4-ethoxycarbonylphenyl, halogen-lower alkylphenyl, typically 4-chloromethylphenyl, imidazol-1-ylphenyl, typically 4-(imidazolyl-1-yl)phenyl, pyrrolidino-lower alkylphenyl, typically 4-pyrrolidinomethylphenyl, piperazino-lower alkylphenyl, typically 4-piperazinomethylphenyl, (4-lower alkylpiperazinomethyl)phenyl, typically 4-(4-methylpiperazinomethyl)phenyl, morpholino-lower alkylphenyl, typically 4-morpholinomethylphenyl, piperazinocarbonylphenyl, typically 4-piperazinocarbonylphenyl, or (4-lower alkylpiperazino)phenyl, typically 4-(4-methylpiperazino)phenyl.

A further preferred Acyl Ac¹ is derived from monoesters of carbonic acid and is characterised by the subformula R°O—CO—. The lower alkyl radicals, especially tert-butyl, are especially preferred hydrocarbyl radicals R° in these derivatives.

Another preferred Acyl Ac¹ is derived from amides of carbonic acid (or also thiocarbonic acid) and is characterised by the formula R°HN—C(═W)— or R°R°N—C(═W)—, wherein the radicals R° are, independently of one another, as defined above and W is sulfur and especially oxygen. In particular, compounds are preferred wherein Ac¹ is a radical of formula R°HN—C(═W)—, wherein W is oxygen and R° has one of the following preferred meanings: morpholino-lower alkyl, typically 2-morpholinoethyl, phenyl, lower alkoxyphenyl, typically 4-methoxyphenyl or 4-ethoxyphenyl, carboxyphenyl, typically 4-carboxyphenyl, or lower alkoxycarbonylphenyl, typically 4-ethoxycarbonylphenyl.

A preferred acyl Ac² of subformula R°—SO₂—, wherein R° is a hydrocarbyl as defined in the above general and specific meanings, is lower alkylphenylsulfonyl, typically 4-toluenesulfonyl.

If p is 0, the nitrogen atom bonding R₃ is uncharged. If p is 1, then R₄ must also be present, and the nitrogen atom bonding R₃ and R₄ (quaternary nitrogen) is then positively charged.

The definitions for an aliphatic, carbocyclic, or carbocyclic-aliphatic radical with up to 29 carbon atoms each, or for a heterocyclic or heterocyclic-aliphatic radical with up to 20 carbon atoms each and up to 9 heteroatoms each, or acyl with up to 30 carbon atoms each, preferably match the definitions given for the corresponding radicals R₃ and R₄. Especially preferred is R₅ lower alkyl, especially methyl, or most especially hydrogen.

Z is especially lower alkyl, most especially methyl.

If the two bonds indicated by wavy lines are missing in ring A, then no double bonds (tetra-hydrogenated derivatives) are present between the carbon atoms characterised in formula I by the numbers 1, 2, 3, and 4, but only single bonds, whereas ring B is aromatic (double bonds between the carbon atoms characterised in formula I by 8 and 9 and those characterised by 10 and 11). If the two bonds indicated by wavy lines are missing in ring B, then no double bonds (tetra-hydrogenated derivatives) are present between the carbon atoms characterised in formula I by the numbers 8, 9, 10, and 11, but only single bonds, whereas ring A is aromatic (double bonds between the carbon atoms characterised in formula I by 1 and 2 and those characterised by 3 and 4). If the total of four bonds indicated by wavy lines are missing in rings A and B, and are replaced by a total of 8 hydrogen atoms, then no double bonds (octa-hydrogenated derivatives) are present between the carbon atoms numbered 1, 2, 3, 4, 8, 9, 10, and 11 in formula I, but only single bonds.

By their nature, the compounds of the invention may also be present in the form of pharmaceutically, i.e. physiologically, acceptable salts, provided they contain salt-forming groups. For isolation and purification, pharmaceutically unacceptable salts may also be used. For therapeutic use, only pharmaceutically acceptable salts are used, and these salts are preferred.

Thus, compounds of formula I having free acid groups, for example a free sulfo, phosphoryl or carboxyl group, may exist as a salt, preferably as a physiologically acceptable salt with a salt-forming basic component. These may be primarily metal or ammonium salts, such as alkali metal or alkaline earth metal salts, for example sodium, potassium, magnesium or calcium salts, or ammonium salts with ammonia or suitable organic amines, especially tertiary monoamines and heterocyclic bases, for example triethylamine, tri-(2-hydroxyethyl)-amine, N-ethylpiperidine or N,N′-dimethylpiperazine.

Compounds of the invention having a basic character may also exist as addition salts, especially as acid addition salts with inorganic and organic acids, but also as quaternary salts. Thus, for example, compounds which have a basic group, such as an amino group, as a substituent may form acid addition salts with common acids. Suitable acids are, for example, hydrohalic acids, e.g. hydrochloric and hydrobromic acid, sulfuric acid, phosphoric acid, nitric acid or perchloric acid, or aliphatic, alicyclic, aromatic or heterocyclic carboxylic or sulfonic acids, such as formic, acetic, propionic, succinic, glycolic, lactic, malic, tartaric, citric, fumaric, maleic, hydroxymaleic, oxalic, pyruvic, phenylacetic, benzoic, p-aminobenzoic, anthranilic, p-hydroxybenzoic, salicylic, p-aminosalicylic acid, pamoic acid, methanesulfonic, ethanesulfonic, hydroxyethanesulfonic, ethylenedisulfonic, halobenzenesulfonic, toluenesulfonic, naphthalenesulfonic acids or sulfanilic acid, and also methionine, tryptophan, lysine or arginine, as well as ascorbic acid.

In view of the close relationship between the novel compounds (especially of formula I) in free form and in the form of their salts, including those salts that can be used as intermediates, for example in the purification or identification of the novel compounds, and of their solvates, any reference hereinbefore and hereinafter to the free compounds is to be understood as referring also to the corresponding salts, and the solvates thereof, for example hydrates, as appropriate and expedient.

The compounds of formula A, B, C, D, I, II, III, IV, V or VI especially those wherein R₅ is hydrogen, possess valuable pharmacological properties.

In the case of the groups of radicals or compounds mentioned hereinbefore and hereinafter, general definitions may, insofar as appropriate and expedient, be replaced by the more specific definitions stated hereinbefore and hereinafter.

Preference is given to a compounds of formula I, II, III, IV, V, VI wherein
R₁ and R₂ independently of each other are lower alkyl, lower alkyl substituted by halogen, C₆-C₁₄aryl, hydroxy, lower alkoxy, phenyl-lower alkoxy, phenyloxy, lower alkanoyloxy, benzoyloxy, amino, lower alkylamino, lower alkanoylamino, phenyl-lower alkylamino, N,N-di-lower alkylamino, N,N-di-(phenyl-lower alkyl)amino, cyano, mercapto, lower alkylthio, carboxy, lower alkoxycarbonyl, carbamoyl, N-lower alkylcarbamoyl, N,N-di-lower alkyl-carbamoyl, sulfo, lower alkanesulfonyl, lower alkoxysulfonyl, aminosulfonyl, N-lower-alkylaminosulfonyl or N,N-di-lower alkylaminosulfonyl; halogen; lower alkoxy; C₆-C₁₄aryloxy; C₆-C₁₄aryl-lower alkoxy; lower alkanoyloxy; C₆-C₄arylcarbonyloxy; amino monosubstituted or disubstituted by lower alkyl, C₆-C₁₄aryl, C₆-C₁₄aryl-lower alkyl, lower alkanoyl or C₆-C₁₂arylcarbonyl; cyano; nitro; mercapto; lower alkylthio; C₆-C₁₄arylthio; C₆-C₁₄aryl-lower alkylthio; lower alkanoylthio; C₆-C₁₄aryl-lower alkanoylthio; carboxy; lower alkoxycarbonyl, C₆-C₁₄aryl-lower alkoxycarbonyl; C₆-C₁₄aryloxycarbonyl; carbamoyl; carbamoyl N-mono- or N,N-disubstituted by lower alkyl, C₆-C₁₄aryl or C₆-C₁₄aryl-lower alkyl; sulfo; C₆-C₁₄arylsulfonyl; C₆-C₁₄aryl-lower alkanesulfonyl; lower alkanesulfonyl; or aminosulfonyl N-mono- or N,N-disubstituted by lower alkyl, C₆-C₁₄aryl or C₆-C₁₄aryl-lower alkyl, wherein C₆-C₁₄aryl is an aryl radical with 6 to 12 carbon atoms in the ring system, which may be unsubstituted or substituted by halogen, phenyl or naphthyl, hydroxy, lower alkoxy, phenyl-lower alkoxy, phenyloxy, lower alkanoyloxy, benzoyloxy, amino, lower alkylamino, lower alkanoylamino, phenyl-lower alkylamino, N,N-di-lower alkylamino, N,N-di-(phenyl-lower alkyl)amino, cyano, mercapto, lower alkylthio, carboxy, lower alkoxycarbonyl, carbamoyl, N-lower alkylcarbamoyl, N,N-di-lower alkylcarbamoyl, sulfo, lower alkanesulfonyl, lower alkoxysulfonyl, aminosulfonyl, N-lower alkylaminosulfonyl or N,N-di-lower alkylaminosulfonyl;
n and m are independently of each other 0 or 1 or 2, preferably 0;
R₃, R₄, R₈, R₁₀ are independently of each other hydrogen, lower alkyl, lower alkenyl or lower alkadienyl, which are each unsubstituted or monosubstituted or polysubsituted, preferably monosubstituted or disubstituted by a substituent independently selected from lower alkyl; hydroxy; lower alkoxy, which may be unsubstituted or mono-, di-, or trisubstituted by (i) heterocyclyl with 4 to 12 ring atoms, which may be unsaturated, wholly saturated, or partly saturated, is monocyclic or bicyclic and may contain up to three heteroatoms selected from nitrogen, oxygen and sulfur, and is most especially pyrrolyl, for example 2-pyrrolyl or 3-pyrrolyl, pyridyl, for example 2-, 3- or 4-pyridyl, or in a broader sense also thienyl, for example 2- or 3-thienyl, or furyl, for example 2-furyl, indolyl, typically 2- or 3-indolyl, quinolyl, typically 2- or 4-quinolyl, isoquinolyl, typically 3- or 5-isoquinolyl, benzofuranyl, typically 2-benzofuranyl, chromenyl, typically 3-chromenyl, benzothienyl, typically 2- or 3-benzothienyl; imidazolyl, typically 1- or 2-imidazolyl, pyrimidinyl, typically 2- or 4-pyrimidinyl, oxazolyl, typically 2-oxazolyl, isoxazolyl, typically 3-isoxazolyl, thiazolyl, typically 2-thiazolyl, benzimidazolyl, typically 2-benzimidazolyl, benzoxazolyl, typically 2-benzoxazolyl, quinazolyl, typically 2-quinazolinyl, 2-tetrahydrofuryl, 4-tetrahydrofuryl, 4-tetrahydropyranyl, 1-, 2- or 3-pyrrolidyl, 1-, 2-, 3-, or 4-piperidyl, 1-, 2- or 3-morpholinyl, 2- or 3-thiomorpholinyl, 2-piperazinyl or N,N′-bis-lower alkyl-2-piperazinyl, (ii) by halogen, (iii) by hydroxy or (iv) by lower alkoxy; phenoxy; phenyl-lower alkoxy; heterocyclyloxy, wherein heterocyclyl is pyrrolyl, for example 2-pyrrolyl or 3-pyrrolyl, pyridyl, for example 2-, 3- or 4-pyridyl, or in a broader sense also thienyl, for example 2- or 3-thienyl, or furyl, for example 2-furyl, indolyl, typically 2- or 3-indolyl, quinolyl, typically 2- or 4-quinolyl, isoquinolyl, typically 3- or 5-isoquinolyl, benzofuranyl, typically 2-benzofuranyl, chromenyl, typically 3-chromenyl, benzothienyl, typically 2- or 3-benzothienyl; imidazolyl, typically 1- or 2-imidazo-lyl, pyrimidinyl, typically 2- or 4-pyrimidinyl, oxazolyl, typically ²-oxazolyl, isoxazolyl, typically 3-isoxazolyl, thiazolyl, typically 2-thiazolyl, benzimidazolyl, typically 2-benzimida-zolyl, benzoxazolyl, typically 2-benzoxazolyl, quinazolyl, typically 2-quinazolinyl, 2-tetrahydrofuryl, 4-tetrahydrofuryl, 2- or 4-tetrahydropyranyl, 1-, 2- or 3-pyrrolidyl, 1-, 2-, or 4-piperidyl, 1-, 2- or 3-morpholinyl, 2- or 3-thiomorpholinyl, 2-piperazinyl or N,N′-bis-lower alkyl-2-piperazinyl, such as especially 2- or 4-tetrahydropyranyloxy; lower alkanoyloxy; carboxy; lower alkoxycarbonyl; phenyl-lower alkoxycarbonyl; mercapto; lower alkylthio; phenylthio; halogen; halogen-lower alkyl; oxo (except in the 1-position, because otherwise acyl); azido; nitro; cyano; amino; mono-lower alkylamino; di-lower alkylamino; pyrrolidino; imidazol-1-yl; piperidino; piperazino; 4-lower alkylpiperazino; morpholino; thiomorpholino; diphenylamino or dibenzylamino unsubstituted or substituted in the phenyl part by lower alkyl, lower alkoxy, halogen and/or nitro; lower alkoxycarbonylamino; phenyl-lower alkoxycarbonylamino unsubstituted or substituted in the phenyl part by lower alkyl or lower alkoxy; fluorenylmethoxycarbonylamino; amino-lower alkyl; monosubstituted or disubstituted amino-lower alkyl, wherein the amino substituent is selected from lower alkyl, hydroxy-lower alkyl, C₃-C₈cycloalkyl, amino-lower alkyl, N-mono- or N,N-di(-lower-alkyl)amino-lower alkyl, amino, N-mono- or N,N-di-lower alkylamino and N-mono- or N,N-di-(hydroxy-lower alkyl)amino; pyrrolidino-lower alkyl; piperidino-lower alkyl; piperazino-lower alkyl; 4-lower alkylpiperazino-lower alkyl; imidazol-1-yl-lower alkyl; morpholino-lower alkyl; thiomorpholino-lower alkyl; S-oxo-thiomorpholino-lower alkyl; S,S-dioxothiomorpholino-lower alkyl; lower alkylendioxy; sulfamoyl; sulfo; carbamoyl; ureido; guanidino; cyano; aminocarbonyl (carbamoyl) and aminocarbonyloxy, which are substituted by one or two radicals on the nitrogen, wherein the amino substituents are selected independently of one another from the group comprising lower alkyl, hydroxy-lower alkyl, C₃-C₈cycloalkyl, amino-lower alkyl, N-mono- or N,N-di(-lower alkyl)amino-lower alkyl, amino, N-mono- or N,N-di-lower alkylamino and N-mono- or N,N-di-(hydroxy-lower alkyl)-amino; pyrrolidinocarbonyl; piperidinocarbonyl; piperazinocarbonyl; 4-lower alkylpiperazinocarbonyl; imidazolinocarbonyl; morpholinocarbonyl; thio-morpholinocarbonyl; S-oxo-thiomorpholinocarbonyl; and S,S-dioxothiomorpholino;
phenyl, naphthyl, phenyl-lower alkyl or phenyl-lower alkenyl with a terminal phenyl radical, which is unsubstituted or monosubstituted or disubstituted by the radicals named above as substituents of lower alkyl, lower alkenyl or lower alkadienyl;
or heterocyclyl-lower alkyl, wherein heterocyclyl is pyrrolyl, for example 2-pyrrolyl or 3-pyrrolyl, pyridyl, for example 2-, 3- or 4-pyridyl, or in a broader sense also thienyl, for example 2- or 3-thienyl, or furyl, for example 2-furyl, indolyl, typically 2- or 3-indolyl, quinolyl, typically 2- or 4-quinolyl, isoquinolyl, typically 3- or 5-isoquinolyl, benzofuranyl, typically 2-benzofuranyl, chromenyl, typically 3-chromenyl, benzothienyl, typically 2- or 3-benzothienyl; imidazolyl, typically 1- or 2-imidazolyl, pyrimidinyl, typically 2- or 4-pyrimidinyl, oxazolyl, typically 2-oxazolyl, isoxazolyl, typically 3-isoxazolyl, thiazolyl, typically 2-thiazolyl, benzimidazolyl, typically 2-benzimidazolyl, benzoxazolyl, typically 2-benzoxazolyl, quinazolyl, typically 2-quinazolinyl, 2-tetrahydrofuryl, 4-tetrahydrofuryl, 2- or 4-tetrahydropyranyl, 1-, 2- or 3-pyrrolidyl, 1-, 2-, 3-, or 4-piperidyl, 1-, 2- or 3-morpholinyl, 2- or 3-thiomorpholinyl, 2-piperazinyl or N,N′-bis-lower alkyl-2-piperazinyl, which in each case are unsubstituted or monosubstituted or disubstituted by the radicals named above as substituents of lower alkyl, lower alkenyl, or lower alkadienyl;
whereby R₄ may also be absent:
or
R₄ is absent, and
R₃ is acyl of the subformulae Y—C(═W)—, wherein W is oxygen and Y is hydrogen, R°, R°—O—, R°HN—, or R°R°N— (wherein the radicals R° may be the same or different),
or
is acyl of the subformula R°—SO₂—,
wherein R° in the said radicals has the following meanings: substituted or unsubstituted lower alkyl, especially methyl or ethyl, amino-lower alkyl hydroxy-lower alkyl, wherein the amino group is unprotected or is protected by a conventional amino protecting group—especially by lower alkoxycarbonyl, typically tert-lower alkoxycarbonyl, for example tert-butoxycarbonyl—e.g. aminomethyl, R,S—,R— or preferably S-1-aminoethyl, tert-butoxycarbonylaminomethyl or R,S—,R—, or preferably S-1-(tert-butoxycarbonylamino)ethyl, carboxy-lower alkyl, typically 2-carboxyethyl, lower alkoxycarbonyl-lower alkyl, typically 2-(tert-butoxycarbonyl)ethyl, cyano-lower alkyl, typically 2-cyanoethyl, tetrahydropyranyloxy-lower alkyl, typically 4-(tetrahydropyranyl)oxymethyl, morpholino-lower alkyl, typically 2-(morpholino)ethyl, phenyl, lower alkylphenyl, typically 4-methylphenyl, lower alkoxyphenyl, typically 4-methoxyphenyl, imidazolyl-lower alkoxyphenyl, typically 4-[2-(imidazol-1-yl)ethyl)oxyphenyl, carboxyphenyl, typically 4-carboxyphenyl, lower alkoxycarbonylphenyl, typically 4-ethoxycarbonylphenyl or 4-methoxyphenyl, halogen-lower alkylphenyl, typically 4-chloromethylphenyl, pyrrolidinophenyl, typically 4-pyrrolidinophenyl, imidazol-1-ylphenyl, typically 4-(imidazolyl-1-yl)phenyl, piperazinophenyl, typically 4-piperazinophenyl, (4-lower alkylpiperazino)phenyl, typically 4-(4-methylpiperazino)phenyl, morpholinophenyl, typically 4-morpholinophenyl, pyrrolidino-lower alkylphenyl, typically 4-pyrrolidinomethylphenyl, imidazol-1-yl-lower alkylphenyl, typically 4-(imidazolyl-1-ylmethyl)phenyl, piperazino-lower alkylphenyl, typically 4-piperazinomethylphenyl, (4-lower alkylpiperazinomethyl)-phenyl, typically 4-(4-methylpiperazinomethyl)phenyl, morpholino-lower alkylphenyl, typically 4-morpholinomethylphenyl, piperazinocarbonylphenyl, typically 4-piperazinocarbonylphenyl, or (4-lower alkylpiperazino)phenyl, typically 4-(4-methylpiperazino)phenyl.

p is 0 if R₄ is absent, or is 1 if R₃ and R₄ are both present and in each case are one of the aforementioned radicals;
R₅ is hydrogen or lower alkyl, especially hydrogen,
X stands for 2 hydrogen atoms, for O, or for 1 hydrogen atom and hydroxy; or for 1 hydrogen atom and lower alkoxy;
Z is hydrogen or especially lower alkyl, most especially methyl;
and either the two bonds characterised by wavy lines are preferably absent in ring A and replaced by 4 hydrogen atoms, and the two wavy lines in ring B each, together with the respective parallel bond, signify a double bond;
or also the two bonds characterised by wavy lines are absent in ring B and replaced by a total of 4 hydrogen atoms, and the two wavy lines in ring A each, together with the respective parallel bond, signify a double bond;
or both in ring A and in ring B all of the 4 wavy bonds are absent and are replaced by a total of 8 hydrogen atoms;
or a salt thereof, if at least one salt-forming group is present.

Particular preference is given to a compound of formula I wherein
m and n are each 0;
R₃ and R₄ are independently of each other
hydrogen,
lower alkyl unsubstituted or mono- or disubstituted, especially monosubstituted, by radicals selected independently of one another from carboxy; lower alkoxycarbonyl; and cyano; whereby R₄ may also be absent;
or
R₄ is absent, and
R₃ is acyl from the subformula R°—CO, wherein R° is lower alkyl, especially methyl or ethyl; amino-lower alkyl, wherein the amino group is unprotected or protected by lower alkoxy-carbonyl, typically tert-lower alkoxycarbonyl, for example tert-butoxycarbonyl, e.g. aminomethyl, R,S—,R—, or preferably S-1-aminoethyl, tert-butoxycarbonylaminomethyl or R,S—,R—, or preferably S-1-(tert-butoxycarbonylamino)ethyl; tetrahydropyranyloxy-lower alkyl, typically 4-(tetrahydropyranyl)oxymethyl; phenyl; imidazolyl-lower alkoxyphenyl, typically 4-[2-(imidazol-1-yl )ethyl)oyxphenyl; carboxyphenyl, typically 4-carboxyphenyl; lower alkoxycarbonylphenyl, typically 4-methoxy- or 4-ethoxycarbonylphenyl; halogen-lower alkylphenyl, typically 4-chloromethylphenyl; imidazol-1-ylphenyl, typically 4-(imidazolyl-1-yl)phenyl; pyrrolidino-lower alkylphenyl, typically 4-pyrrolidinomethylphenyl; piperazino-lower alkylphenyl, typically 4-piperazinomethylphenyl; (4-lower alkylpiperazinomethyl)phenyl, typically 4-(4-methylpiperazinomethyl)phenyl; morpholino-lower alkylphenyl, typically 4-morpholinomethylphenyl; piperazinocarbonylphenyl, typically 4-piperazinocarbonylphenyl; or (4-lower alkylpiperazino)phenyl, typically 4-(4-methylpiperazino)phenyl;
or is acyl of the subformula R°—O—CO—, wherein R° is lower alkyl;
or is acyl of the subformula R°HN—C(═W)—, wherein W is oxygen and R° has the following preferred meanings: morpholino-lower alkyl, typically 2-morpholinoethyl, phenyl, lower alkoxyphenyl, typically 4-methoxyphenyl or 4-ethoxyphenyl, carboxyphenyl, typically 4-carboxyphenyl, or lower alkoxycarbonylphenyl, typically 4-ethoxycarbonylphenyl;
or is lower alkylphenylsulfonyl, typically 4-toluenesulfonyl;
p is 0 if R₄ is absent, or is 1 if R₃ and R₄ are both present and in each case are one of the aforementioned radicals;
R₅ is hydrogen or lower alkyl, especially hydrogen,
X stands for 2 hydrogen atoms or for O;
Z is methyl;
and either the two bonds characterised by wavy lines are preferably absent in ring A and replaced by 4 hydrogen atoms, and the two wavy lines in ring B each, together with the respective parallel bond, signify a double bond;
or also the two bonds characterised by wavy lines are absent in ring B and replaced by a total of 4 hydrogen atoms, and the two wavy lines in ring A each, together with the respective parallel bond, signify a double bond;
or both in ring A and in ring B all of the 4 wavy bonds are absent and are replaced by a total of 8 hydrogen atoms;
or a salt thereof, if at least one salt-forming group is present.

Most especially preferred is the compound of formula I designated 1,2,3,4-tetrahydro-staurosporine, or a (particularly pharmaceutically acceptable) salt thereof (here, m und n in formula I are 0, R₃ is hydrogen, R₄ is absent, provided no salt is present (p=0), or is hydrogen if a salt is present (p=1), R₅ is hydrogen, the two bonds represented by wavy lines are absent in Ring A and are replaced by a total of 4 hydrogen atoms and the two bonds represented by wavy lines in Ring B are in each case a double bond together with the parallel bonds, X stands for 2 hydrogen atoms, and Z is methyl).

Most especially preferred are the compounds of formula A wherein
X═O; R₁, R₂, R₅═H; Q═—(CH₂)₂—O—CH(CH₂)OH—(CH₂)₂— (LY 333531)
X═O; R₁, R₂, R₅═H; Q═—(CH₂)₂—O—CH(CH₂N(CH₃)₂)—(CH₂)₂—
X=2 hydrogen atoms; R₁, R₂, R₅═H;

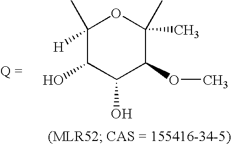

Most especially preferred are the compounds of formula I wherein;
X=2 hydrogen atoms; R₁, R₂, R₃, R₅═H; R₄═CH₃; Z═CH₃ (staurosporine)
X=1 hydrogen and 1 hydroxy atoms in (R) or (S) isomeric form; R₁, R₂, R₃, R₅═H; R₄═CH₃;
Z═CH₃ (UCN-01 and UCN-02)
X=2 hydrogen atoms; R₁, R₂, R₅═H; R₄═CH₃; R₃=benzoyl; Z═CH₃ (CGP41251 or PKC412 or midostaurin)
X═O; R₁, R₂, R₅═H; R₃═CH₃; R₄=ethyloxycarbonyl; Z═CH₃ (NA 382—CAS=143086-33-3)
E) X=1 hydrogen and 1 hydroxy atom; R₁, R₂, R₅═H; R₃═CH₃; Z═CH₃; and R₄ is selected from —(CH₂)₂OH; —CH₂CH(OH)CH₂OH; —CO(CH₂)₂CO₂Na; —(CH₂)₃CO₂H;
—COCH₂N(CH₃)₂; 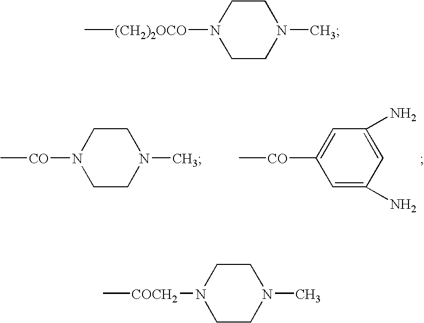
X=2 hydrogen atoms; R₁, R₂, R₅═H; R₃═CH₃; Z═CH₃; and R₄ is selected from N—[0-(tetrahydropyran-4-yl )-D-lactoyl]; N-[2-methyl-2-(tetrahydropyran-4-yloxy)-propionyl; N-[0-(tetrahydropyran-4-yl )-L-lactoyl]; N-[0-(tetrahydropyran-4-yl )-D-lactoyl]; N-[2-(tetrahydro-pyran-4-yloxy)-acetyl)]
X═O; R₁, R₂, R₅═H; R₃═CH₃; Z═CH₃; and R₄ is selected from N-[0-(tetrahydropyran-4-yl)-D-lactoyl]; N-[2-(tetrahydro-pyran-4-yloxy)-acetyl)]
X═1 hydrogen and 1 hydroxy atom; R₁, R₂, R₅═H; R₃═CH₃; Z═CH₃; and R₄ is selected from N-[0-(tetrahydropyran-4-yl )-D-lactoyl]; N-[2-(tetrahydro-pyran-4-yloxy)-acetyl)]

The most preferred compound I also known as MIDOSTAURIN [International Nonproprietary Name] has been specifically described in the European patent No. 0 296 110 published on Dec. 21, 1988, as well as in U.S. Pat. No. 5,093,330 published on Mar. 3, 1992, and Japanese Patent No. 2 708 047 all in the name of the applicant.

Most especially preferred are the compounds of formula III wherein
X=2 hydrogen atoms; R₁, R₂, R₅═H; R₆═CH₃; R₇=methyloxycarbonyl; Z═H (2-methyl K252a)
X=2 hydrogen atoms; R₁, R₂, R₅, R₆═H; R₇=methyloxycarbonyl; Z═H (K-252a)
X=2 hydrogen atoms; R₁, R₂, R₅, R₆═H; R₇=methyloxycarbonyl; Z=CH₃ (KT-5720)

Most especially preferred are the compounds of formula IV wherein;
X═O; R₁, R₂, R₅═H; R₉═CH₂—NMe₂; R₈═CH₃; m′=n′=2
X═O; R₁, R₂, R₅═H; R₀═CH₂—NH₂; R₈═CH₃; m′=2; n′=1 (Ro-31-8425; CAS=151342-35-7)

Most especially preferred are the compounds of formula V wherein;
X═O; R₁, R₂, R₅═H; R₈═CH₃; R₁₀═—(CH₂)₃—NH₂; (Ro-31-7549; CAS=13851-31)
X═O; R₁, R₂, R₅═H; R₈═CH₃; R₁₀═—(CH₂)₃—S—(C═NH)—NH₂; (Ro-31-8220, CAS=125314-64-9))
X═O; R₁, R₂, R₅═H; R₈═CH₃; R₁₀═CH₃;

Most especially preferred are the compounds of formula VI wherein;
A) X=2 hydrogen atoms; R₁, R₂, R₅═H; R₄═CH₃; Z═CH₃; R₃ selected from methyl or (C₁-C₁₀)alkyl, arylmethyl, C₆H₂CH₂—

STAUROSPORINE DERIVATIVES and their manufacturing process have been specifically described in many prior art documents, well known by the man skilled in the art.

Compounds of formula A, B, C, D and their manufacturing process have for instance, been described in the European patents No. 0 657 458 published on Jun. 14, 1995, in the European patents No. 0 624 586 published on Nov. 17, 1994, in the European patents No. 0 470 490 published on Feb. 12, 1992, in the European patents No. 0 328 026 published on Aug. 16, 1989, in the European patents No. 0 384 349 published on Aug. 29, 1990, as well as in many publications such as Barry M. Trost and Weiping Tang, Org. Lett., 3(21), 3409-3411.

Compounds of formula I and their manufacturing process has been specifically described in the European patents No. 0 296 110 published on Dec. 21, 1988, as well as in U.S. Pat. No. 5,093,330 published on Mar. 3, 1992, and Japanese Patent No. 2 708 047 all in the name of the applicant. Compounds of formula I having a tetrahydropyran-4-yl)-lactoyl substitution on R₄ have been described in the European patent No. 0 624 590 published on Nov. 17, 1994. Other compounds have been described in the European patent No. 0 575 955 published Dec. 29, 1993, European patent No. 0 238 011 published on Sep. 23, 1987 (UCN-O1), International patent application EP98/04141 published as W099/02532 on Jul. 03, 1998.

Compounds of formula II and their manufacturing process has been specifically described in the European patents No. 0 296 110 published on Dec. 21, 1988, as well as in U.S. Pat. No. 5,093,330 published on Mar. 3, 1992, and Japanese Patent No. 2 708 047 all in the name of the applicant.

Compounds of formula III and their manufacturing process has been specifically described in the patent applications claiming the priority of the U.S. patent application U.S. Pat. No. 920,102 filed on Jul. 24, 1992. (i.e. European patents No. 0 768 312 published on Apr. 16, 1997, No. 1 002 534 published May 24, 2000, No. 0 651 754 published on May 10, 1995).

Compounds of formula IV and their manufacturing process has been specifically described in the patent applications claiming the priority of the British patent applications GB 9309602 and GB 9403249 respectively filed on May 10, 1993, and on Feb. 21, 1994. (i.e. European patents No. 0 624 586 published on Nov. 17, 1994, No. 1 002 534 published May 24, 2000, No. 0 651 754 published on May 10, 1995).

Compounds of formula V and their manufacturing process has been specifically described in the patent applications claiming the priority of the British patent applications GB 8803048, GB 8827565, GB 8904161 and GB 8928210 respectively filed on Feb. 10, 1988, Nov. 25, 1988, Feb. 23, 1989 and Dec. 13, 1989. (i.e. European patents No. 0 328 026 published on Aug. 16, 1989, and No. 0 384 349 published Aug. 29, 1990).

Compounds of formula VI and their manufacturing process has been specifically described in the patent applications claiming the priority of the U.S. patent applications Ser. No. 07/777,395 (Con), filed on Oct. 10, 1991 (i.e. International patent application WO 93/07153 published on Apr. 15, 1993)

STAUROSPORINE DERIVATIVES were originally identified as inhibitor of protein kinase C (PKC) (Meyer T, Regenass U, Fabbro D, et al. Int. J. Cancer 43: 851-856, 1989).

Compound I of the following formula 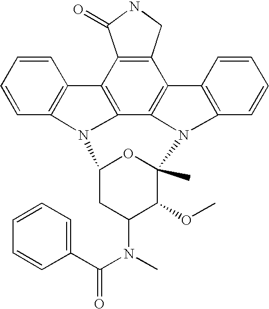
is a staurosporine analog and has been shown to potently inhibit conventional protein kinase C isoforms α, β, γ and the calcium-independent protein kinase C isoforms δ, ε and ηin the nanomolar concentrations.

Compound I is currently being developed as an anticancer agent. Phase I and II studies with Compound I show that this orally available agent is safe with minimal toxicity. Oral doses at 150 to 300 mg/day in patients with advanced cancer resulted in serum levels of Compound I that are in the micromolar range.

The term “agents effective in preventing, delaying or treating transplant rejection” as used herein relates to a macrocyclic lactone having immunosuppressive properties, a mTOR inhibitor, e.g. rapamycin, 40-O-(2-hydroxyethyl)-rapamycin (RAD), CCI779 or ABT578 or a rapalog disclosed, e.g. in WO 98/02441 or WO 01/14387, e.g. AP23573; an ascomycin having immunosuppressive properties, e.g. ABT-281, ASM981, etc.; cyclophosphamide, azathioprine; methotrexate; leflunomide; mizoribine; mycophenolic acid or a salt thereof, e.g. Myfortic®, mycophenolate mofetil; a somatostatin analogue like octreotide, lanreotide, vapreotide or SOM230, a calcineurin inhibitor, e.g. cyclosporin A, FK 506 or cyclosporins having a cis or trans unsaturated lateral chain in position 1, e.g. as disclosed in EP-B1-0296 122; a deoxyspergualine compound or derivative or analog thereof, e.g. 15-DSG, corticosteroids, e.g., prednisolone, methylprednisolone and dexamethasone, monoclonal antibodies to leukocyte receptors, e.g., MHC, CD2, CD3, CD4, CD7, CD8, CD11a/CD18, CD25, CD27, CD28, CD40. CD45, CD58, CD80, CD86, CD134, CD137, ICOS, CD150 (SLAM), CD152, OX40, 4-1BB or to their ligands, e.g. CD154, or antagonists thereof; other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a homologue or a mutant thereof, e.g. LEA29Y; adhesion molecule inhibitors, e.g. LFA-1 antagonists, ICAM-1 or -3 antagonists, anti-LFA-1 or anti-ICAM antibodies, VCAM-4 antagonists or VLA-4 antagonists; or anti-chemokine antibodies or anti-chemokine receptor antibodies or low molecular weight chemokine receptor antagonists, e.g. anti MCP-1 antibodies. Preferably, the agent effective in preventing, delaying or treating transplant rejection is a calcineurin inhibitor, most preferably cyclosporin A.

The structure of the active agents cited and identified by code numbers, generic or trademark names may be taken from the actual edition of the standard compendium “The Merck Index” or from databases, e.g. Patents International (e.g. IMS World Publications). The corresponding content thereof is hereby incorporated by reference. Any person skilled in the art is fully enable, based on these references, to manufacture and test the pharmaceutical indications and properties in standard test models, both in vitro and in vivo.

The compounds used as combination partners (a) and (b) disclosed herein can be prepared and administered as described in the cited documents, respectively or according to the manufacturer instructions.

It will be understood that references to the combination partners (a) and (b) are meant to also include the pharmaceutically acceptable salts. If these combination partners (a) and (b) have, for example, at least one basic center, they can form acid addition salts, e.g. succinates. Corresponding acid addition salts can also be formed having, if desired, an additionally present basic center. The combination partners (a) and (b) having an acid group (for example COOH) can also form salts with bases. The combination partner (a) or (b) or a pharmaceutically acceptable salt thereof may also be used in form of a hydrate or include other solvents used for crystallization.

A combination which comprises (a) and (b) in which the active ingredients are present in each case in free form or in the form of a pharmaceutically acceptable salt and optionally at least one pharmaceutically acceptable carrier, will be referred to hereinafter as a COMBINATION OF THE INVENTION.

The COMBINATIONS OF THE INVENTION inhibit the transplant rejection.

All the more surprising is the experimental finding that in vivo the administration of a COMBINATION OF THE INVENTION compared to a monotherapy applying only one of the pharmaceutically active ingredients used in the COMBINATION OF THE INVENTION results not only in a more beneficial, especially synergistic, e.g. anti-transplant rejection effect, e.g. with regard to delaying transplant rejection, but also in further surprising beneficial effects, e.g. less side-effects and a decreased mortality and morbidity. The COMBINATIONS OF THE INVENTION are also suitable to prevent the transplant rejection.

A further benefit is that lower doses of the active ingredients of the COMBINATION OF THE INVENTION can be used, for example, that the dosages need not only often be smaller but are also applied less frequently, or can be used in order to diminish the incidence of side-effects observed with one of the combination partners alone. This is in accordance with the desires and requirements of the patients to be treated.

It can be shown by established test models that a COMBINATION OF THE INVENTION results in the beneficial effects described herein before. The person skilled in the pertinent art is fully enabled to select a relevant test model to prove such beneficial effects. The pharmacological activity of a COMBINATION OF THE INVENTION may, for example, be demonstrated in a clinical study or in a test procedure as essentially described hereinafter.

Suitable clinical studies are, for example, open labeled non-randomized, placebo-controlled, parallel studies in transplanted patients. Such studies are, in particular, suitable to compare the effects of a monotherapy using the active ingredients and a therapy using a COMBINATION OF THE INVENTION, and to prove in particular the synergism of the active ingredients of the COMBINATIONS OF THE INVENTION. The primary endpoints in such studies can be the effect on pain scores, analgesic use, performance status, Quality of Life scores or time to progression of the disease.

It is one objective of this invention to provide a pharmaceutical composition comprising a quantity, which is jointly therapeutically effective against a proliferative disease comprising the COMBINATION OF THE INVENTION. In this composition, the combination partners (a) and (b) can be administered together, one after the other or separately in one combined unit dosage form or in two separate unit dosage forms. The unit dosage form may also be a fixed combination.

The pharmaceutical compositions for separate administration of the combination partners (a) and (b) and for the administration in a fixed combination, i.e. a single galenical composition comprising at least two combination partners (a) and (b), according to the invention can be prepared in a manner known per se and are those suitable for enteral, such as oral or rectal, and parenteral administration to patients, comprising a therapeutically effective amount of at least one pharmacologically active combination partner alone or in combination with one or more pharmaceutically acceptable carriers, especially suitable for enteral or parenteral application.

The novel pharmaceutical composition contains, for example, from about 10% to about 100%, preferably from about 20% to about 60%, of the active ingredients. Pharmaceutical preparations for the combination therapy for enteral or parenteral administration are, for example, those in unit dosage forms, such as sugar-coated tablets, tablets, capsules or suppositories, and furthermore ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In particular, the combination partners of the COMBINATION OF THE INVENTION may be administered simultaneously or sequentially and in any order, and the components may be administered separately or as a fixed combination. For example, the method of delaying or treating a transplant rejection according to the invention may comprise (i) administration of the first combination partner in free or pharmaceutically acceptable salt form and (ii) administration of the second combination partner in free or pharmaceutically acceptable salt form, simultaneously or sequentially in any order, in jointly therapeutically effective amounts, preferably in synergistically effective amounts, e.g. in daily dosages corresponding to the amounts described herein. The individual combination partners of the COMBINATION OF THE INVENTION can be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimes of simultaneous or alternating treatment and the term “administering” is to be interpreted accordingly.

The effective dosage of each of the combination partners employed in the COMBINATION OF THE INVENTION may vary depending on the particular compound or pharmaceutical composition employed, the mode of administration, the condition being treated, the severity of the condition being treated. Thus, the dosage regimen the COMBINATION OF THE INVENTION is selected in accordance with a variety of factors including the route of administration and the renal and hepatic function of the patient. A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites. This involves a consideration of the distribution, equilibrium, and elimination of the active ingredients.

The STAUROSPORINE DERIVATIVES can be administered parenterally, e.g., intra-peritoneally, intravenously, intramuscularly, subcutaneously, intra-tumorally, or rectally, or enterally, e.g., orally, preferably intravenously or orally, intravenously at a daily dosage of 1-300 mg/kg body weight in patients. A preferred daily dosage is 1-75 mg/kg body weight. In human trials, a total dose of 225 mg/day was most presumably the Maximum Tolerated Dose (MTD) for a patient of about 70 kg.

There is a relationship between Cyclosporin A blood levels and clinical outcome as measured by toxicity and the rate of rejection. Low levels of Cyclosporin A are associated with increased rates of rejection and high levels associated with increased rates of toxicity. Because of this, measurement of Cyclosporin A blood levels is a standard of care and the dosing of Cyclosporin A is adjusted in patients according to said blood levels. The management of Cyclosporin A administration to patient is well known by the person skilled in the art.

When the combination partners employed in the COMBINATION OF THE INVENTION are applied in the form as marketed as single drugs, their dosage and mode of administration can take place in accordance with the information provided on the packet leaflet of the respective marketed drug in order to result in the beneficial effect described herein, if not mentioned herein otherwise.

The COMBINATION OF THE INVENTION can be a combined preparation or a pharmaceutical composition.

Moreover, the present invention relates to a method of treating a recipient patient of organ, tissue or cell transplant having or susceptible to develop transplant rejection comprising administering to said patient a COMBINATION OF THE INVENTION in a quantity which is jointly therapeutically effective against transplant rejection and in which the combination partners can also be present in the form of their pharmaceutically acceptable salts. In one embodiment of the invention, in such method the COMBINATION OF THE INVENTION is co-administered with an anti-emetic agent.

The invention also relates to a method of inhibiting acute or chronic transplant rejection mechanism in a patient having a transplant which comprises administering to said patient a pharmaceutically effective amount of the COMBINATION OF THE INVENTION in a quantity which is jointly therapeutically effective against said transplant rejection and in which the compounds can also be present in the form of their pharmaceutically acceptable salts.

Furthermore, the present invention pertains to the use of a COMBINATION OF THE INVENTION for the delay of progression or treatment of an acute or chronic transplant rejection and for the preparation of a medicament for the delay of progression or treatment of an acute or chronic transplant rejection.

Moreover, the present invention provides a commercial package comprising as active ingredients COMBINATION OF THE INVENTION, together with instructions for simultaneous, separate or sequential use thereof in the delay of progression or treatment of acute or chronic transplant rejection.

The following non limitative examples illustrate the invention.

### EXAMPLE 1

### Mixed Lymphocyte Reaction

The two-way MLR is performed according to standard procedures (J. Immunol. Methods, 1973, 2, 279 and Meo T. et al., Immunological Methods, New York, Academic Press, 1979, 227-39). Briefly, spleen cells from CBA and BALB/c mice (1.6×10⁵ cells from each strain per well in flat bottom tissue culture microtiter plates, 3.2×10⁵ in total) are incubated in RPMI medium containing 10% FCS, 100 U/ml penicillin, 100 μg/ml streptomycin (Gibco BRL, Basel, Switzerland), 50 μM 2-mercaptoethanol (Fluka, Buchs, Switzerland) and serially diluted compounds. Seven three-fold dilution steps in duplicates per test compound are performed. After four days of incubation 1 μCi ³H-thymidine is added. Cells are harvested after an additional five-hour incubation period, and incorporated ³H-thymidine is determined according to standard procedures. Background values (low control) of the MLR are the proliferation of BALB/c cells alone. Low controls are subtracted from all values. High controls without any sample are taken as 100% proliferation. Percent inhibition by the samples is calculated, and the concentrations required for 50% inhibition (IC₅₀ values) are determined.

### EXAMPLE 2

### Compound I and CsA Inhibit Lectin Induced Mononuclear Cell Proliferation

Peripheral blood mononuclear cells (PBMCs) are isolated from buffy coats obtained from the Stanford University Medical Center Blood Bank by Ficoll centrifugation. PBMCs are incubated with varying concentrations of Compound I with and without cyclosporin A for 30 minutes prior to the addition of concanavalin A (ConA, 7.5 μg/ml) in a 96 well microtiter plate (1×10⁵ cells/well). Cells are plated in quadruplicate. After a 54 hour incubation, cells are pulsed with ³H-thymidine (0.1 μCi/well) and cells are harvested onto a glass filtermat 18 hours later. Scintillation fluid is added and filtermats are quantitated with microplate counter.

| ConA | − | + | + | + | + | + | + | + | + | + | + | + | + |
|---|---|---|---|---|---|---|---|---|---|---|---|---|---|
| CsA | − | − | 50 | 100 | − | − | − | 50 | 50 | 50 | 100 | 100 | 100 |
| CI | − | − | − | − | 0.05 | 0.1 | 1 | 0.05 | 0.1 | 1 | 0.05 | 0.1 | 1 |
| cpm | 1860 | 14321 | 10136 | 9102 | 11944 | 9005 | 2556 | 8472 | 5260 | 1082 | 7307 | 3162 | 642 |
| SD | 721 | 546 | 703 | 1481 | 1996 | 1321 | 1198 | 359 | 528 | 608 | 482 | 1754 | 263 |

CsA is the cyclosporin A concentration given in ng/ml, C I corresponds to Compound I concentrations given in mM, cpm are the values of the means in count per minutes for 5 samples, SD is the standard deviation.

Compound I inhibits ConA induced PBMC proliferation in a concentration dependent manner with an IC₅₀ of approximately 0.4 μM. The inhibitory effects of Compound I are additive to those of CsA; the IC₅₀ of Compound I with 50 ng/ml CsA is 0.06 μM.

Those in vitro results show that the immunomodulatory effect of Compound I is additive to those of CsA, and thus a combination of these two agents may permit dose reduction of CsA and avoidance of toxicity associated with this medication. In conclusion, Compound I alone or the combination of Compound I and CsA has potent immunomodulatory activity and is a promising candidate as an anti-rejection agent.

### EXAMPLE 3

### Rat Heart Transplantation

The animals are anaesthetised using inhalational isofluorane. Following heparinisation of the donor rat through the abdominal inferior vena cava with simultaneous exsanguination via the aorta, the chest is opened and the heart rapidly cooled. The aorta is ligated and divided distal to the first branch and the brachiocephalic trunk is divided at the first bifurcation. The left pulmonary artery is ligated and divided and the right side divided but left open. All other vessels are dissected free, ligated and divided and the donor heart is removed into iced saline. The recipient is prepared by dissection and cross-clamping of the infra-renal abdominal aorta and vena cava. The graft is implanted with end-to-side anastomoses, using 10/0 monofilament suture, between the donor brachiocephalic trunk and the recipient aorta and the donor right pulmonary artery to the recipient vena cava. The clamps are removed, the graft tethered retro-abdominally, the abdominal contents washed with warm saline and the animal is closed and allowed to recover under a heating lamp. Graft survival is monitored by daily palpation of the beating donor heart through the abdominal wall. Rejection is considered to be complete when heart beat stops.

### EXAMPLE 4

### Compositions

Examples of useful compositions comprising compounds of formula I are described in the European patents No. 0 296 110, No. 0 657 164, No. 0 296 110, No.0 733 372, No.0 711 556, No.0 711 557, all in the name of the applicant.

The preferred compositions are described in the European patent No. 0 657 164 published on Jun. 14, 1995. The described pharmaceutical compositions comprise a solution or dispersion of compounds of formula I such as MIDOSTAURIN in a saturated polyalkylene glycol glyceride, in which the glycol glyceride is a mixture of glyceryl and polyethylene glycol esters of one or more C₈-C₁₈ saturated fatty acids.

Two manufacture processes of such compositions are described hereafter.

Composition A:

Gelucire 44/14 (82 parts) is melted by heating to 60° C. Powdered Compound 1 (18 parts) is added to the molten material. The resulting mixture is homogenised and the dispersion obtained is introduced into hard gelatin capsules of different size, so that some contain a 25 mg dosage and others a 75 mg dosage of Compound I. The resulting capsules are suitable for oral administration.

Composition B:

Gelucire 44/14 (86 parts) is melted by heating to 60° C. Powdered Compound 1 (14 parts) is added to the molten material. The mixture is homogenised and the dispersion obtained is introduced into hard gelatin capsules of different size, so that some contain a 25 mg dosage and others a 75 mg dosage of Compound I. The resulting capsules are suitable for oral administration.

Gelucire 44/14 available commercially from Gattefossé, is a mixture of esters of C₈-C₁₈ saturated fatty acids with glycerol and a polyethylene glycol having a molecular weight of about 1500, the specifications for the composition of the fatty acid component being, by weight, 4-10% caprylic acid, 3-9% capric acid, 40-50% lauric acid, 14-24% myristic acid, 4-14% palmitic acid and 5-15% stearic acid.

A preferred example of Gelucire formulation consists of:
Gelucire (44/14): 47 g
Compound I: 3.0 g filled into a 60 ml Twist off flask

A preferred example of soft gel will contain the following microemulsion:

| | |
|---|---|
| Cornoil glycerides | 5.00 mg |
| Polyethylenglykol 400 | 128.25 mg |
| Cremophor RH 40 | 213.75 mg |
| Compound 1 | 25.00 mg |
| DL alpha Tocopherol | 0.50 mg |
| Ethanol absolute | 33.90 mg |
| Total | 486.40 mg |

### EXAMPLE 5

The effects of Compound I (midostaurin) on lymphocyte proliferation induced by lectin or irradiated allogeneic cells are measured by [³H] thymidine uptake. The effects of Compound I on levels of T cell activation surface markers CD25 and CD71 are measured by flow cytometry after lectin. Intracellular calcium response to lectin stimulation is measured in Compound I treated peripheral blood mononuclear cells by flow cytometry and the ratiometric calcium indicator dyes fluo-4 and Fura-red. Intracellular IL-2, TNF-alpha, IFN-gamma expression in Compound I treated T cells stimulated by lectin is measured by flow cytometry.

Materials and Methods:

Quantitation of lectin induced mononuclear cell proliferation. Peripheral blood mononuclear cells (PBMC's) are isolated from buffy coats obtained from the Stanford University Medical Center Blood Bank by Ficoll centrifugation. PBMC's are incubated with varying concentrations of Compound I with and without cyclosporine for 30 minutes prior to the addition of concanavalin A (ConA, 7.5 μg/ml) in a 96 well microtiter plate (1×10⁵ cells/well). Cells are plated in quadruplicate. After a 54 hour incubation, cells are pulsed with [³H] thymidine (0.1 μCi/well) and cells are harvested onto a glass filtermat 18 hours later. Scintillation fluid is added and filtermats are quantitated with microplate counter. Quantitation of T cell surface antigen expression. Human PBMC's are treated with varying concentrations of Compound I for 30 minutes prior to stimulation with ConA (7.5 μg/ml). After 72 hours incubation in growth conditions, surface activation markers CD25 and CD71 are measured on CD3⁺ T cells.

Intracellular Calcium Kinetics. Human PBMC's are resuspended in calcium-free PBS and loaded with the calcium indicator dyes Fura-red and fluo-4 for 1 hour at room temperature shielded from light. Compound I at varying concentrations is also added at this time. At the end of dye loading, cells are washed in PBS and resuspended in RPMI 1640 and placed in growth conditions. One million cells of each sample are then analyzed by a flow cytometer and the mean FL-1 and FL-3 channels recorded every 8 seconds for a total of 240 seconds. At least eight thousand events are acquired during each eight-second interval. PHA (20 μg/ml) was added to activate T cells at time 0 seconds. The ratio FL-1/FL-3 is directly related to the intracellular calcium concentration and is expressed in arbitrary units (AU).

Quantitation of intracellular T cell cytokine expression. PBMC's are treated with varying concentrations of Compound I for 30 minutes prior to stimulation with ConA (15 μg/ml) in the presence of brefeldin for 6 hours and then incubated with anti-CD3□-PerCP mAb. Cells are then fixed, permeabilized and incubated with anti-cytokine (IL-2, TNF-α and IFN-γ) mAbs.

Flow cytometric analysis. All samples are assessed by three color analysis flow cytometry (Epics XL-MCL/498 nm air cooled argon laser Beckman-Coulter) within 6 hours of preparation. Forward-scatter gates are used to differentiate cell of interest from debris and 5,000 light scatter gated T or 3,000 B lymphocytes or 3,000 monocytes are analyzed per sample. For controls, mAbs are added to unstimulated blood processed in parallel and inactive immunoglobulin isotype controls are added to cultures of stimulated cells.

Results: Compound I inhibits lectin and allogeneic cell induced lymphocyte proliferation with an IC₅₀ of 0.25 to 0.4 mM. Compound I inhibits the upregulation of CD25 and CD71 in a concentration dependent fashion with IC₅₀'s similar to those seen in the lymphocyte proliferation assays. Compound I blunts the intracellular calcium response of peripheral blood mononuclear cells to PHA at micromolar concentrations. Compound I potently inhibits TNF-alpha production by CD3⁺ T cells with an IC₅₀ of 0.5 mM. Compound I slightly increases IL-2 and IFN-gamma production by CD3+ T cells at concentrations less than 0.5 mM and inhibited the production of these cytokines at greater concentrations with an IC₅₀ in the low micromolar range.

Compound I potently inhibits T lymphocyte activation and function and may be a useful adjuvant immunosuppressive agent in the field of organ transplantation.

1. A combination which comprises (a) a staurosporine derivative and (b) an agent effective in preventing, delaying or treating transplant rejection in which the active ingredients (a) and (b) are present in each case in free form or in the form of a pharmaceutically acceptable salt and optionally at least one pharmaceutically acceptable carrier; for simultaneous, separate or sequential use.

2. The combination according to claim 1 wherein (a) is of formula 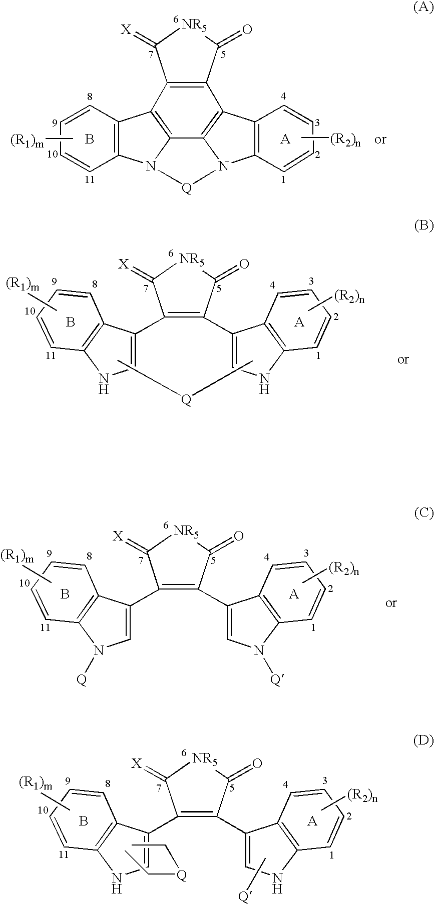
wherein R₁, and R₂ are, independently of one another, unsubstituted or substituted alkyl, hydrogen, halogen, hydroxy, etherified or esterified hydroxy, amino, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl or N-mono- or N,N-di-substituted aminosulfonyl;
n and m are, independently of one another, a number from and including 0 to and including 4;
R₅ is hydrogen, an aliphatic, carbocyclic, or carbocyclic-aliphatic radical with up to 29 carbon atoms in each case, or a heterocyclic or heterocyclic-aliphatic radical with up to 20 carbon atoms in each case, and in each case up to 9 heteroatoms, or acyl with up to 30 carbon atoms;
X stands for 2 hydrogen atoms; for 1 hydrogen atom and hydroxy; for O; or for hydrogen and lower alkoxy;
Q and Q′ are independently a pharmaceutically acceptable organic bone or hydrogen, halogen, hydroxy, etherified or esterified hydroxy, amino, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl or N-mono- or N,N-di-substituted aminosulfonyl;
or a salt thereof, if at least one salt-forming group is present, or hydrogenated derivative thereof.

3. The combination according to claim 2 wherein the staurosporine derivative is selected from the compounds of formula 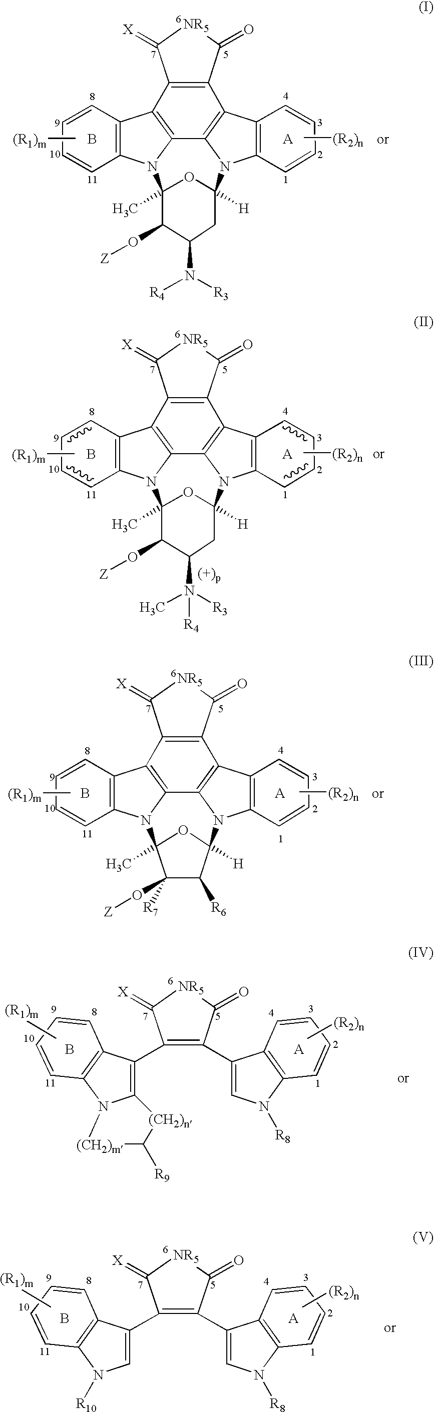 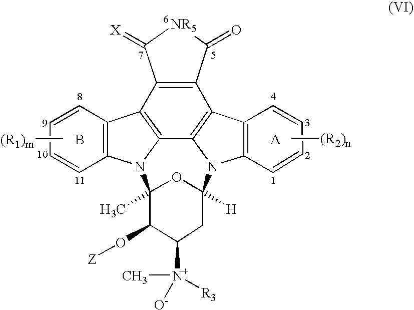
wherein R₁ and R₂, are, independently of one another, unsubstituted or substituted alkyl, hydrogen, halogen, hydroxy, etherified or esterified hydroxy, amino, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl or N-mono- or N,N-di-substituted aminosulfonyl;
n and m are, independently of one another, a number from and including 0 to and including 4;
n′ and m′ are, independently of one another, a number from and including 1 to and including 4;
R₃, R₄, R₈ and R₁₀ are, independently of one another, hydrogen, an aliphatic, carbocyclic, or carbocyclic-aliphatic radical with up to 29 carbon atoms in each case, a heterocyclic or heterocyclic-aliphatic radical with up to 20 carbon atoms in each case, and in each case up to 9 heteroato-ms, an acyl with up to 30 carbon atoms, wherein R₄ may also be absent;
or R₃ is acyl with up to 30 carbon atoms and R₄ not an acyl;
p is 0 if R₄ is absent, or is 1 if R₃ and R₄ are both present and in each case are one of the aforementioned radicals;
R₅ is hydrogen, an aliphatic, carbocyclic, or carbocyclic-aliphatic radical with up to 29 carbon atoms in each case, or a heterocyclic or heterocyclic-aliphatic radical with up to 20 carbon atoms in each case, and in each case up to 9 heteroatoms, or acyl with up to 30 carbon atoms;
R₇, R₆ and R₉ are acyl or -(lower alkyl)-acyl, unsubstituted or substituted alkyl, hydrogen, halogen, hydroxy, etherified or esterified hydroxy, amino, mono- or disubstituted amino, cyano, nitro, mercapto, substituted mercapto, carboxy,carbonyl, carbonyldioxy, esterified carboxy, carbamoyl, N-mono- or N,N-di-substituted carbamoyl, sulfo, substituted sulfonyl, aminosulfonyl or N-mono- or N,N-di-substituted aminosulfonyl;
X stands for 2 hydrogen atoms; for 1 hydrogen atom and hydroxy; for O; or for hydrogen and lower alkoxy;
Z stands for hydrogen or lower alkyl;
and either the two bonds characterised by wavy lines are absent in ring A and replaced by 4 hydrogen atoms, and the two wavy lines in ring B each, together with the respective parallel bond, signify a double bond;
or the two bonds characterised by wavy lines are absent in ring B and replaced by a total of 4 hydrogen atoms, and the two wavy lines in ring A each, together with the respective parallel bond, signify a double bond;
or both in ring A and in ring B all of the 4 wavy bonds are absent and are replaced by a total of 8 hydrogen atoms;
or a salt thereof, if at least one salt-forming group is present.

4. The combination according to claim 3 wherein in the compound of formula I, X=2 H; R₁, R₂ and R₅═H; R₄ and Z═CH₃; and R₃ is benzoyl (midostaurin).

5. The combination according to claim 1 wherein (b) is selected from the group consisting of calcineurin inhibitors.

6. The combination according to claim 5 wherein the calcineurin inhibitor is cyclosporin A.

7. A method of preventing or treating acute or chronic transplant rejection in a recipient patient of organ or tissue or cell transplant comprising the step of administering to said patient a therapeutically effective amount of a staurosporine derivative.

8. (canceled)

9. The method according to claim 17 wherein the compound of formula I is midostaurin.

10. A method of treatment of acute or chronic transplant rejection of a patient having a transplant, comprising administering to said patient a combination according to claim 1 in a quantity which is jointly therapeutically effective against transplant rejection and in which the compounds can also be present in the form of their pharmaceutically acceptable salts.

11. The method of treatment according to claim 9 wherein midostaurin is administered at a dosage of 1-75 mg/kg body weight/day.

12. A pharmaceutical composition comprising a quantity which is jointly therapeutically effective against transplant rejection of a pharmaceutical combination according to claim 1 and at least one pharmaceutically acceptable carrier.

13. A pharmaceutical composition as a fixed combination comprising (a) a staurosporine derivative and (b) an agent effective against transplant rejection.

14. The pharmaceutical composition according to claim 12 wherein (a) is midostaurin and (b) is cyclosporin A.

15. A pharmaceutical composition containing an amount of midostaurin for use in the treatment of acute or chronic transplant rejection

16. A commercial package comprising a pharmaceutical composition according claim 15 together with instructions for simultaneous, separate or sequential use thereof in the treatment of transplant rejection.

17. A method of preventing or treating acute or chronic transplant rejection in a recipient patient of organ or tissue or cell transplant comprising the step of administering to said patient a therapeutically effective amount of a staurosporine derivative wherein the staurosporine derivative is the compound of formula I according to claim 3.
